SEQUENCE LISTING (2 sequences)

SEQ ID NO: 1 - DNA, 27 bp, Artificial Sequence
  LAP gene forward amplification primer
gaccatggatggcaattaaagaaaatg

SEQ ID NO: 2 - DNA, 25 bp, Artificial Sequence
  Reverse LAP gene amplification primer
gactcgagtcaaacacctttgtaag

(19) United States
(12) Patent Application Publication
(10) Pub. No.: US 2013/0202571 A1
(43) Pub. Date: Aug. 8, 2013

(54) USE OF PROBIOTIC BACTERIA TO PREVENT AND TREAT LISTERIAL INFECTIONS

(71) Applicant: Purdue Research Foundation, West Lafayette, IN (US)

(72) Inventors: Arun K. BHUNIA, West Lafayette, IN (US); Ok Kyung Koo, Fayetteville, AR (US); Mary-Anne Roshni Amalaradjou, West Lafayette, IN (US)

(73) Assignee: Purdue Research Foundation, West Lafayette, IN (US)

(21) Appl. No.: 13/755,483

(22) Filed: Jan. 31, 2013

Related U.S. Application Data
(60) Provisional application No. 61/594,143, filed on Feb. 2, 2012.

(51) Int. Cl. A61K 35/74

(52) U.S. Cl. CPC A61K 35/747; A61K 35/741
USPC 424/93.45; 424/93.4; 435/252.3

(57) ABSTRACT

This invention relates to LAP-expressing probiotic bacteria and methods of use thereof to prevent and treat a pathogenic bacterial infection.

### CROSS-REFERENCE TO RELATED APPLICATIONS

This application claims priority of U.S. Provisional Application Ser. No. 61/594,143 filed 2 Feb. 2012. This application is hereby incorporated in its entirety by reference herein.

### FIELD OF THE INVENTION

This invention relates to LAP-expressing probiotic bacteria and methods of use thereof to prevent and treat a pathogenic bacterial infection.

### BACKGROUND OF INVENTION

Listeria monocytogenes causes a severe systemic infection (listeriosis) and poses a significant health risk to pregnant women, newborns, the elderly, and other immunocompromised individuals.

Annually, about 2,500 Americans contract invasive listeriosis with a mortality rate of 20-30%. Traditional vaccination is not economical for the treatment and control of listeriosis owing to the small number of cases. Listeriosis is predominantly contracted through contaminated food, although neonatal listeriosis is acquired from the mother. During the gastrointestinal phase of infection, intestinal epithelial cells and M (microfold) cells are the primary sites of interaction. Adhesion, invasion, and translocation across the intestinal epithelial barrier are a prerequisite for pathogenesis. Therefore, devising strategies to block the initial site of pathogen interaction is an effective and logical approach to protecting hosts against enteric infections.

LAP is an alcohol acetaldehyde dehydrogenase (lmo1634), a housekeeping enzyme with a molecular mass of about 104 kDa. It interacts strongly with host cells of intestinal origin and binds to host cell receptor Hsp60. More specifically, the N2 domain (Gly224-Gly411) in the N-terminus of LAP interacts with Hsp60. Surface expression and secretion of LAP depend on SecA2, an auxiliary secretion system present in Gram-positive bacteria. Our previous studies have demonstrated that LAP expression is enhanced in oxygen- and nutrient-limited conditions and at elevated temperatures (37-42° C.). In the intestine, L. monocytogenes crosses the epithelial barrier by invading epithelial cells through the intracellular route using Internalin (InlA or InlB) proteins. Listeria can also cross the epithelial barrier via the paracellular route. Interaction of LAP with Hsp60 compromises the tight junction barrier, allowing increased paracellular translocation of L. monocytogenes. Furthermore, L. monocytogenes translocation occurs independently of InlA: an inlA mutant strain translocated efficiently through the epithelial barrier.

Probiotic bacteria are regarded as safe and have been used to promote human health. These bacteria colonize and proliferate in the intestine, producing metabolites and macromolecules with beneficial effects including health maintenance and prevention or alleviation of enteric infection, allergic diseases, and chronic inflammatory diseases such as inflammatory bowel disease, Crohn's disease, and ulcerative colitis. The use of probiotics to prevent and treat infections is gaining attention as a substitute for antibiotic or anti-inflammatory drugs because antibiotic resistance and the emergence of “superbugs” threaten public health. One of the most critical functions of probiotics is infection prevention, likely mediated by increased defensin production, induction of anti-inflammatory responses, suppression of pro-inflammatory cytokines (i.e., tumor necrosis factor alpha, interleukin [IL]-8, IL-6), increased production of shortchain fatty acids (butyrate) during fermentation, and improved epithelial tight junction barrier function. Probiotic bacteria attach to intestinal cells via electrostatic or hydrophobic interactions, steric forces, lipoteichoic acids, or specific surface proteins and prevent pathogen binding through a mechanism referred to as steric hindrance. Probiotic bacterial cells, cell wall components such as S-layer proteins, and secretory compounds are also known to prevent enteric pathogen colonization and neutralize toxins. Although many enteric diseases have been controlled by probiotics, the approach has had limited success or been ineffective with L. monocytogenes. Furthermore, the normal anti-pathogen adhesive activity of probiotics is often unpredictable and unsatisfactory and may be unsuitable for inhibiting the attachment of specific pathogens to a host.

A need exists for novel and effective strategies to inhibit the initial attachment of Listeria to host cells in order to minimize infection. Doing so would prevent listeriosis in susceptible populations. The invention provided herein fulfills this need by providing a recombinant probiotic strain expressing LAP to competitively exclude adhesion, transepithelial translocation, and cell cytotoxicity of L. monocytogenes.

### SUMMARY OF THE INVENTION

In one embodiment, the invention relates to a recombinant probiotic bacteria strain expressing Listeria adhesion protein (LAP).

In one embodiment, the invention relates to a method of preventing a LAP-expressing pathogenic bacterial infection in a subject, the method comprising the step of administering to the subject a prophylactically-effective dose of an LAP-expressing probiotic bacterial strain.

In one embodiment, the invention relates to a method of delivering a foreign protein into a host cell in a subject, the method comprising administering to the subject a recombinant LAP-expressing probiotic strain comprising or expressing said foreign protein.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The subject matter regarded as the invention is particularly pointed out and distinctly claimed in the specification. The invention, however, may best be understood by reference to the following detailed description when read with the accompanying drawings in which:

FIG. 1 shows adhesion profile of lactic acid bacteria to Caco-2 cells. Bacteria were added to Caco-2 cells at a ratio of 10:1. Percent adhesion was calculated relative to the inoculums that were added to the Caco-2 cells for the adhesion assay. The data are average ±standard deviation (SD) of two independent experiments performed in triplicate. Bars marked with letters (a, b, c, d) are significantly different at P<0.05.

FIG. 2 shows Listeria adhesion protein (LAP) expression analysis in recombinant Lactobacillus paracasei (Lbp^{LAP}). (a) Plasmid map (10.6 kb) of LAP expression vector pLP401T (9.8 kb)-LAP (2.6 kb). Ery, erythromycin resistance gene; Amp, ampicillin resistance gene; Ori+=origin of replication of E. coli, Ori-=origin of replication of Lactobacillus; LAP, Listeria adhesion protein; Pamy, a-amylase promoter gene; ssAmy, secretion signal (36 aa) and the N-terminus (26 aa) of a-amylase gene; Anchor, anchor peptide (117 aa) gene of Lb. casei; Tcbh, transcription terminator of the cbh (conjugated bile acid hydrolase) gene; Rep, repA gene. (b) Western blot showing LAP expression in the supernatant, cell wall, and intracellular fractions of Listeria monocytogenes (Lm) and Lbp^{LAP} but absent in wild type Lb. paracasei (Lbp^{WT}). Molecular weight of LAP from Lm and the recombinant Lbp^{LAP} was similar. (c) Immunofluorescence staining of bacteria (magnification 1000×) with anti-LAP MAb-H7 and fluorescein isothiocyanate-conjugated second antibody (left panel) and Hoechst dye (blue; right panel). Lbp^{LAP} and Lm (control) cells indicated the presence of surface-expressed LAP (green) that was absent in Lbp^{WT}. (d) Binding (capture) of recombinant Lb. paracasei cells to paramagnetic beads coated with Hsp60 relative to L. monocytogenes (considered 100%). The data are average (SD) of two independent experiments performed in duplicate. Letters (a,b) indicate significant difference at P<0.05.

FIG. 3 shows Adhesion characteristics of recombinant Lactobacillus paracasei (Lbp^{LAP}) to Caco-2 cells. (a) Adhesion of Lbp^{LAP} was compared with wild type Lb. paracasei (Lbp^{WT}) and L. monocytogenes (Lm). Bacterial cells were incubated with anti-LAP MAb-H7 or immunoglobulin G controls (MAb EM-7G1) (1 mg/ml) for 30 min at room temperature, washed and added to Caco-2 cells. The number of bacterial cells that adhered to the monolayer were enumerated. Percent adhesion was calculated relative to the inoculums that were added to the Caco-2 cells for the adhesion assay. Data are average (SD) of three independent experiments performed in duplicate. Bars marked with letters (a, b) indicate significant difference at P<0.05. (b) Representative Giemsa-stained Caco-2 cell monolayers showing visual evidence for qualitative adhesion characteristics of Lbp^{WT} and Lbp^{LAP} cells. Bar, 10 μm.

FIG. 4 shows (a) Translocation and (b) internalization of recombinant Lactobacillus paracasei (Lbp^{LAP}) and wild type Lb. paracasei (Lbp^{WT}). In the translocation assay, Caco-2 cells were grown on transwell filter inserts for 10-12 days to differentiate and to reach confluence. Bacteria were added to the apical well of the insert and incubated for 2 h. Liquid from the basal well was removed and plated for bacterial enumeration. In the invasion assay, bacteria were added to Caco-2 cells at an MOE of 10:1/well in 24-well tissue culture plates and incubated for 1 h. After washing (3×), Caco-2 cells were incubated in D10F containing 50 mg/mL gentamicin, lysed using 0.1% Triton-X 100 and intracellular bacteria were enumerated following plating. The data are average (SD) of three independent experiments analyzed in duplicate.

FIG. 5 shows Competitive exclusion of Listeria monocytogenes (Lm) adhesion to Caco-2 cells by recombinant Lactobacillus paracasei (Lbp^{LAP}), analyzed by three adhesion methods. (a) competitive adhesion: Caco-2 cells were exposed to wild type Lb. paracasei (Lbp^{WT}) or Lbp^{LAP} with Lm simultaneously, (b) inhibition of adhesion: Caco-2 cells were pre-exposed to Lbp^{WT} or Lbp^{LAP} for 1 h before infection with Lm, and (c) displacement experiments: Caco-2 cells were infected with Lm for 1 h before Lbp^{WT} or Lbp^{LAP} addition. Adhesion of (Lm) alone to Caco-2 cells was presented as 100%. Lap-deficient LmKB208 was used as a negative control in the competitive adhesion assay. The data are average (SD) of three independent experiments performed in duplicate. Bars marked with letters (a, b) indicate significant difference at P<0.05.

FIG. 6 shows inhibition of Listeria monocytogenes (Lm) adhesion, invasion, and transepithelial translocation by recombinant Lactobacillus paracasei (Lbp^{LAP}). Caco-2 cells were exposed to Lbp^{LAP}, Lbp^{LAP}—(vector without LAP insert) or wild type (Lbp^{WT}) for 1, 4, 15, and 24 h before infection with Lm for 1 h in (a) adhesion and (b) invasion experiments, and 2 h for (c) transepithelial translocation experiments. Data are averages of three experiments run in triplicate. Bars marked with letters (a, b, c, d) are significantly different at P<0.05. Table below each graph shows average log Lm counts (SD) for each treatment.

FIG. 7 shows microscopic analysis of protection of Caco-2 cells from Listeria monocytogenes (Lm)-mediated damage by recombinant Lb. paracasei (Lbp^{LAP}). Caco-2 cells pre-exposed to wild type Lb. paracasei (Lbp^{WT}) or Lbp^{LAP} for 15 h before infection with Lm for 1 h were stained with a mixture of acridine orange (green) for live cells and propidium iodide (red) for dead cells. Orange-red cells in the merged picture indicate dead or dying cells. Bar, 10 μm.

FIG. 8 shows competitive exclusion analysis of Listeria monocytogenes by different Lactobacillus species to Caco-2 cells. Three adhesion methods were used; (a) competitive adhesion, (b) inhibition of adhesion, and (c) displacement. First bar shows adhesion of L. monocytogenes to Caco-2 cells without pretreatment of LAB and presented as 100%. Tables (a1, b1, c1) under bar graph show percent adhesion values of L. monocytogenes with and without Lb. rhamnosus, Lb. acidophilus and Lb. paracasei. Also adhesion of each Lactobacillus species in the presence (w) and absence (w/o) of L. monocytogenes was shown. The data are average (SD) of three independent experiments analyzed in duplicate.

FIG. 9 shows displacement of Listeria monocytogenes adhesion following pretreatment of Caco-2 cells with different (a) lactic acid bacterial (LAB) strains and (b) different ratios of Lactobacillus rhamnosus or Lb. acidophilus to L. monocytogenes. First bar shows adhesion of L. monocytogenes to Caco-2 cells without pretreatment of LAB and presented as 100%. Other bars indicate relative adhesion rate of L. monocytogenes after addition of each LAB. The data are average (SD) of two independent experiments performed in triplicate.

FIG. 10 shows binding (capture) analysis of different lactobacilli to Hsp60 coated paramagnetic beads. First bar shows capture rate of L. monocytogenes to Hsp60-coated beads and presented as 100%. Other bars indicate relative capture rate for other bacteria. The data are average (SD) of two independent experiments performed in duplicate.

FIG. 11 shows adhesion characteristics of bacteria to Caco-2 cells pretreated with anti-Hsp60 antibody. (a) Adhesion of L. monocytogenes to Caco-2 cell monolayers that were pre-treated with anti-Hsp60 monoclonal antibody (1 mg/well for 1 h) or an isotype IgG control antibody (purified MAb C11 E9 specific for L. monocytogenes) followed by exposure to Lbp^{WT}, recombinant Lbp^{LAP}, and a vector control, i.e., Lb. paracasei containing empty vector, pLP401-T without any LAP insert (Lbp^{LAP}-) for 1 h. Adherent bacterial counts were determined by plating following lysis of cells using Triton-X 100. (b) adhesion characteristics of Lbp^{WT} and Lbp^{LAP} to Caco-2 cells pretreated with anti-Hsp60 MAb or an isotype antibody MAb C11E9.

FIG. 12 shows an analysis of Listeria adhesion protein (LAP) expression in recombinant Lactobacillus casei: Western blot showing LAP expression in (a) whole cell protein preparation, (b) cell fractions of Lactobacillus casei expressing LAP of L. monocytogenes (LbcLAP^{Lm}), and Lb. casei expressing LAP of L. innocua (LbcLAP^{Lin}). Purified recombinant LAP of L. monocytogenes (rLAP^{Lm}) was used as a control. (b) Immunofluorescence staining using anti-LAP MAb-H7 to verify LAP expression in cells.

FIG. 13 shows an in vitro cell culture (Caco-2) experiment showing inhibition of L. monocytogenes (a) adhesion (b) invasion, and (c) transepithelial translocation of Listeria monocytogenes by Lactobacillus casei (Lbc) expressing LAP of L. monocytogenes (LbcLAP^{Lm}), Lb. casei expressing LAP of L. innocua (LbcLAP^{Lin}) and Lb. paracasei expressing LAP of L. monocytogenes (LbcLAP^{Lm}: as a positive control from Koo et al 2012). Lm, L. monocytogenes; LbcLAP⁻, Lb. casei carrying empty plasmid vector pLP401T without any insert.

FIG. 14 shows survival of recombinant Lactobacillus casei (LbcLAP^{Lin}; LbcLAP^{Lm}) and LbcWT in (a) simulated gastric fluid (SGF), (b) simulated intestinal fluid I (SGF-I), and (c) simulated intestinal fluid II (SGF-II).

FIG. 15 shows light microscopic photographs showing the live and dead stained recombinant probiotic L. casei expressing LAP of L. innocua (LbcLAP^{Lin}) strain (AKB906) using cFDA-SE (carboxyfluoresceindiacetatesuccinimidyl ester) and PI (propidium iodide) after exposure to (a) simulated gastrointestinal fluid (SGF) for 30 min and (b) simulated intestinal fluid (SIF) for 2.5 h.

FIG. 16 shows a mouse (strain; A/J; female, 8-10 weeks) bioassay with Listeria monocytogenes. (a) Mice experiment outline, (b) assessment of probiotic colonization in the intestine during 10 days of feeding, (c) L. monocytogenes counts in mice samples fed with wild type probiotic (LbcWT) or recombinant probiotic L. casei expressing either LAP from L. monocytogenes (LbcLAP^{Lm}) or L. innocua (LbcLAP^{Lin}) for 10 days before oral gavage with L. monocytogenes (Lm). Recombinant probiotic showed significant reduction in L. monocytogenes counts in all samples after 24-48 h of infection (* P<0.05).

FIG. 17 shows a wild type and recombinant probiotic counts in intestine and fecal samples of mice from day 12. MRS containing vancomycin (300 μg/ml) was used to enumerate L. casei WT (LbcWT) (n=15 mice) and MRS containing erythromycin (2 μg/ml) was used to enumerate recombinant probiotic, LbcLAP^{Lin} (n=15) and LbcLAP^{Lm} (n=15). Probiotics were not detected from control animals or control animals that received L. monocytogenes (Lm) only.

FIG. 18 shows mice body weight (g) during probiotic feeding and challenge with L. monocytogenes (Lm).

FIG. 19 shows animal health status and gross pathological changes observed in cecum, liver and spleen of control and probiotic fed mice after Listeria monocytogenes challenge. Panels are; Control, no probiotics; no probiotic+Lm, mice received no probiotic but challenged with L. monocytogenes; and LbcLAP^{Lin}+Lm, recombinant probiotic fed mice orally challenged with L. monocytogenes.

FIG. 20 shows immunohistopathological staining of ileal tissue with antibody to CD3 for total T cell counts.

FIG. 21 shows levels of secretory IgA (sIgA) in mice intestinal mucus after probiotic feeding.

### DETAILED DESCRIPTION OF THE PRESENT INVENTION

Immunocompromised populations such as pregnant women and their fetuses, infants, the elderly, HIV-infected patients, and patients receiving chemotherapy are most vulnerable to infectious diseases. Increasing concerns about antibiotic resistance, the emergence of superbugs, and the lack of targeted treatments has created interest in using alternative methods against these diseases. One such alternative treatment involves the use of probiotic bacteria. In this respect, the present invention provides recombinant probiotic bacteria expressing genes that are required for pathogen adhesion and colonization in order to prevent infection from LAP-expressing pathogenic bacteria such as L. monocytogenes.

In one embodiment, provided herein is a recombinant probiotic bacteria strain expressing Listeria adhesion protein (LAP). In another embodiment, provided herein is a vaccine to reduce LAP-expressing bacterial infections in high-risk subjects, the vaccine comprising a recombinant probiotic bacteria strain expressing Listeria adhesion protein (LAP).

In one embodiment, the probiotic bacterium is a modified Lactobacillus (Lb) casei. In another embodiment, the Lb. casei expresses LAP from a non-pathogenic bacterium. In another embodiment, the non-pathogenic bacterium is a non-pathogenic Listeria. In another embodiment, the non-pathogenic Listeria is L. innocua.

In one embodiment, provided herein is a method of delivering a foreign protein into a mammalian cell in a subject or in a controlled environment, the method comprising the step of respectively administering to the subject or adding into the subject or controlled environment a predetermined amount of LAP-expressing probiotic bacteria.

In one embodiment, a “predetermined amount” is an amount necessary to achieve a desired effect, such as a prophylactic or therapeutic effect. Such an amount can be empirically determined by the skilled artisan. In another embodiment, a desired effect is to prevent LAP-expressing pathogenic bacteria-induced cell cytotoxicity in a host cell, in a subject or in a controlled environment. In another embodiment, the desired effect is to achieve a reduction of LAP-expressing pathogenic bacteria-induced cell cytotoxicity in a host cell, in a subject or in controlled environment. In another embodiment, the desired effect is to prevent adhesion, invasion and translocation of a LAP-expressing pathogenic bacterium into a host cell in a subject. In another embodiment, the desired effect is to prevent a LAP-expressing pathogenic bacterial infection in a subject.

In one embodiment, the host cell is an epithelial cell. In another embodiment, the epithelial cell is a mammalian epithelial cell. In another embodiment the mammalian epithelial cell is a human cell. In another embodiment, the epithelial cell is an intestinal epithelial cell.

In one embodiment, provided herein is a method of reducing LAP-expressing pathogenic bacteria-induced tight junction permeability of a host cell in a subject, or in a controlled environment, the method comprising the step of respectively administering to the subject or adding into the controlled environment a predetermined amount of LAP-expressing probiotic bacteria.

In one embodiment, the invention relates to a method of reducing LAP-expressing pathogenic bacteria-induced cell cytotoxicity of a cell in a subject or in a controlled environment, the method comprising the step of respectively administering to the subject or adding into the controlled environment a LAP-expressing probiotic bacteria strain.

In one embodiment, provided herein is a method of preventing adhesion, invasion and translocation of a LAP-expressing pathogenic bacteria in a host cell in a subject or in a controlled environment, the method comprising the step of respectively administering to a subject or adding into the controlled environment a predetermined amount of LAP-expressing probiotic bacteria. In one embodiment, blocking the initial adhesion/invasion of pathogenic bacteria such as L. monocytogenes allows control of an infection by the pathogenic bacteria.

In one embodiment, the controlled environment is an in vitro assay. In another embodiment, the controlled environment is an in vivo assay. In another embodiment, the in vitro assay is a competitive exclusion assay. In another embodiment, the in vitro assay is a competitive adhesion assay. In another embodiment, the in vitro assay is a lactate dehydrogenase assay (see Example 5 below). In another embodiment, the in vitro assay is a competitive exclusion assay, an adhesion assay, a competitive adhesion assay, a displacement assay, an inhibition assay, a permeability assay, or a lactate dehydrogenase assay.

In one embodiment, provided herein is a method of preventing a LAP-expressing pathogenic bacterial infection in a subject, the method comprising the step of administering to the subject a prophylactically-effective dose of a LAP-expressing probiotic bacterial strain. In another embodiment, the method of preventing simultaneously additional LAP-expressing bacterial-mediated adhesion to and invasion into a host cell in the subject. In another embodiment, the method of preventing LAP-expressing pathogenic bacteria-induced tight junction permeability of a host cell in a subject. In another embodiment, the method of preventing LAP-expressing pathogenic bacteria-induced cell cytotoxicity.

Structurally, LAP from L. monocytogenes is similar to LAP of non-pathogenic Listeria but due to a defect in surface re-association of LAP on non-pathogens, it is unable to perform LAP-mediated adhesion and transepithelial paracellular translocation. The present invention makes use of this in order to generate a bioengineered probiotic expressing LAP derived from a non-pathogenic Listeria in order to prevent and/or treat listeriosis. In another embodiment, the non-pathogenic Listeria is Listeria innocua.

In one embodiment, the term “Treatment,” covers any treatment of a disease in a mammal, particularly in a human, and includes: (a) reducing the incidence and/or risk of relapse (remission, “flare-up”) of the disease during a symptom-free period; (b) relieving or reducing a symptom of the disease; (c) preventing the disease from occurring in a subject which may be predisposed to the disease but has not yet been diagnosed as having it; (d) inhibiting the disease, i.e., arresting its development (e.g., reducing the rate of disease progression); (e) reducing the frequency of episodes of the disease; and (f) relieving the disease, i.e., causing regression of the disease.

The terms “individual,” “host,” “subject,” and “patient,” used interchangeably herein, refer to a mammal, particularly a human.

In one embodiment, provided herein is a method of treating a LAP-expressing pathogenic bacterial infection in a subject, the method comprising the step of administering to the subject a therapeutically-effective dose of a LAP-expressing probiotic bacterial strain and a therapeutically-effective dose of an antibiotic agent. In another embodiment, the method of treating simultaneously reduces additional LAP-expressing bacterial-mediated adhesion to and invasion into a host cell in the subject while eliminating pathogenic bacteria within the host cell in the subject, thereby treating the bacterial infection. In another embodiment, the method of treating reduces LAP-expressing pathogenic bacteria-induced tight junction permeability of a host cell in a subject. In another embodiment, the method of treating reduces LAP-expressing pathogenic bacteria-induced cell cytotoxicity.

In one embodiment, the invention relates to a method of delivering a foreign protein into a host cell in a subject or in a controlled environment, the method comprising the step of administering to the subject or adding into the controlled environment a predetermined amount of a LAP-expressing probiotic bacterium.

In one embodiment, the subject receiving the probiotic administration provided herein is a subject in need of the same because of disease. In another embodiment, the subject receiving the probiotic administration provided herein is a healthy subject. In cases where the subject is a healthy subject, administration of the probiotic bacteria provided herein serves to prevent disease (prophylactic use) from a pathogenic bacterium such as L. monocytogenes. In another embodiment, the subject is a high-risk host.

In one embodiment, probiotic approach has two fold benefits to targeted immunocompromised population at critical times by providing protection against specific pathogen and providing probiotic-attributed general health benefits.

In one embodiment, the terms “treating”, “therapeutic”, “therapy” are used interchangeably herein and refer to therapeutic treatment, while “inhibiting” and “suppressing” refer to prophylactic or preventative measures, wherein the object is to prevent or lessen the targeted pathologic condition as described hereinabove. Thus, in one embodiment, treating may include directly affecting or curing the disease, disorder or condition and/or related symptoms, while suppressing or inhibiting may include preventing, reducing the severity of, delaying the onset of, reducing symptoms associated with the disease, disorder or condition, or a combination thereof. In one embodiment, “prophylaxis,” “prophylactic,” “preventing” or “inhibiting” refers, inter alia, to delaying the onset of symptoms, preventing relapse to a disease, decreasing the number or frequency of relapse episodes, increasing latency between symptomatic episodes, or a combination thereof. In one embodiment, “suppressing” refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, the probiotic bacterial strain is an LAP-expressing Lactobacillus strain. In another embodiment, the LAP-expressing Lactobacillus strain is Lactobacillus paracasei (Lp-LAP). In another embodiment, the LAP-expressing Lactobacillus strain is Lactobacillus casei (Lbc-LAP). In another embodiment, the Lactobacillus strain is an LAP-expressing Lb. rhamnosus. In another embodiment, the Lactobacillus strain is an LAP-expressing Lb. acidophilus. In another embodiment, LAP is expressed on the surface of the probiotic bacteria. In another embodiment, LAP binds to or interacts with mammalian protein receptor heat shock protein 60 (Hsp60).

In one embodiment, Lb. paracasei is used as the host for generation of the recombinant strain because the Lactobacillus expression vector pLP401-T delivers protein effectively due to the presence of a secretion signal, and the leader sequence of CW proteinase from Lb. paracasei. The fusion of these sequences with heterologous genes permits secretion and surface association of heterologous proteins to the peptidoglycan via anchor encoding sequence, prtP from Lb. casei (see Examples below).

Suitable bacteria for inclusion in the instant formulations include, but are not limited to, bacteria of various species, including lactobacillus species, e.g., Lactobacillus acidophilus, L. plantarum, L. casei, L. rhamnosus, L. delbrueckii (including subspecies bulgaricus), L. reuteri, L. fermentum, L. brevis, L. lactis, L. cellobiosus, L. GG, L. gasseri, L. johnsonii, and L. plantarum; bifidobacterium species, e.g., Bifidobacterium bifidum, B. infantis, B. longum, B. thermophilum, B. adolescentis, B. breve, B. animalis; streptococcus species, e.g., Streptococcus lactis, S. cremoris, S. salivarius (including subspecies thermophilus), and S. intermedius; Leuconostoc species; Pediococcus species; Propionibacterium species; Baci Ilus species; non-enteropathogenic Escherichia species, e.g., non-enteropathogenic Escherichia coli, e.g., E. coli Nissle, and the like; and Enterococcus species such as Enterococcus faecalis, and E. faecium. Other suitable probiotic bacteria are known in the art, and have been described. See, e.g., U.S. Pat. No. 5,922,375. The person skilled in the art would understand and recognize those microorganisms which may be included in the compositions of the invention.

Bacteria other than the bacteria that are commonly considered as probiotic bacteria can also be used in a subject formulation. For example, bacteria that are normally pathogenic when viable can also be used, as long as the bacteria are inactivated before use. In one embodiment, the term “inactivated” refers to non-viable bacteria or bacteria with reduced viability and includes inactivation via heat, ultrasound, irradiation, pasteurization or chemical means.

Viability of bacteria is determined using any known method. For example, bacteria are contacted with a membrane-permeant fluorescent dye (e.g., SYTO 9, SYTOX, and the like) that labels live bacteria with green fluorescence; and membrane-impermeant propidium iodide that labels membrane-compromised bacteria with red fluorescence. Roth et al. (1997) Appl. Environ. Microbiol. 63:2421-2431; Lebaron et al. (1998) Appl. Environ. Microbiol. 64:2697-2700; and Braga et al. (2003) Antimicrob. Agents Chemother. 47:408-412. Bacterial viability is also determined by plating the bacteria on an agar plate containing requisite nutritional supplements, and counting the number of colonies formed (colony forming units, cfu).

As another non-limiting example, a subject probiotic formulation comprises two different Lactobacillus strains, e.g., different isolates of the same species that are genetically diverse. As another non-limiting example, a subject probiotic formulation comprises from one to four Lactobacillus strains and from one to four Bifidobacterium strains. As another non-limiting example, a subject probiotic formulation comprises from one to four Lactobacillus strains, from one to four Bifidobacterium strains, and a non-enteropathogenic E. coli strain. As another non-limiting example, a subject probiotic formulation comprises from one to four Lactobacillus strains and a non-enteropathogenic E. coli strain. As another non-limiting example, a subject probiotic formulation comprises from one to four Bifidobacterium strains, and a non-enteropathogenic E. coli strain.

In another embodiment, the probiotic and pathogenic bacteria provided herein adhere to or colonize host cells in the subject or in a controlled environment. In another embodiment, the host cells are mammalian cells. In another embodiment, the mammalian cells are epithelial cells. In another embodiment, the epithelial cells are intestinal epithelial cells. In another embodiment, the epithelial cells are human intestinal epithelial cells. In another embodiment, the epithelial cells are Caco-2 cells.

In one embodiment, the probiotic bacteria prevent adhesion, trans-epithelial translocation or cell cytotoxicity of LAP-expressing pathogenic bacteria into the epithelial cells. In another embodiment, the probiotic bacteria prevent adhesion, trans-epithelial translocation and cell cytotoxicity of LAP-expressing pathogenic bacteria into the epithelial cells. In another embodiment, the translocation is trans-epithelial translocation. In another embodiment, the translocation is paracellular translocation. In one embodiment, increased translocation is mediated by the specific binding of LAP to Hsp60. In another embodiment, translocated Lactobacilli are rapidly eliminated by the host immune system and thus may not be found even when administered in higher doses.

In one embodiment, the pathogenic bacterium is a Listeria. In another embodiment, the Listeria provided herein is a Listeria monocytogenes.

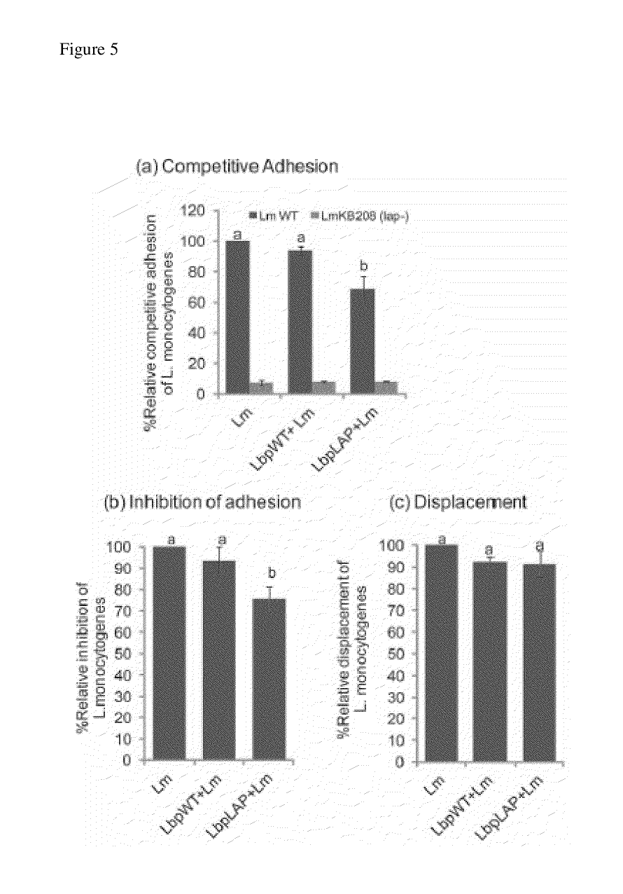

In one embodiment, the recombinant probiotic bacteria provided herein reduce adhesion of pathogenic bacteria to the host cell. In another embodiment, the recombinant probiotic Lactobacilli provided herein reduces adhesion of L. monocytogenes to the host by 31% (see FIG. 5a).

In one embodiment, wild-type Lactobacillus failed to exclude L. monocytogenes adhesion. In another embodiment, Lactobacillus wild-type strains Lb. rhamnosus, Lb. acidophilus, and Lb. paracasei, all with different adhesion abilities, fail to significantly exclude L. monocytogenes adhesion. In one embodiment, the recombinant Lactobacillus provided herein significantly excludes L. monocytogenes adhesion to, invasion of and translocation into an epithelial cell. In another embodiment, the recombinant Lactobacillus provided herein significantly reduced L. monocytogenes-mediated cytotoxicity to an epithelial cell.

In another embodiment, the recombinant probiotic Lactobacillus strain provided herein prevents adhesion of a LAP-expressing pathogenic bacterium by 20-30%. In another embodiment, the recombinant probiotic Lactobacillus strain provided herein prevents adhesion of a LAP-expressing pathogenic bacteria provided herein by 31-40%. In another embodiment, the recombinant probiotic Lactobacillus strain provided herein prevents adhesion of a LAP-expressing pathogenic bacterium by 41-50%. In another embodiment, the recombinant probiotic Lactobacillus strain provided herein prevents adhesion of a LAP-expressing pathogenic bacteria provided herein by 51-60%. In another embodiment, the recombinant probiotic Lactobacillus strain provided herein prevents adhesion of a LAP-expressing pathogenic bacteria provided herein by 61-70%. In another embodiment, the recombinant probiotic Lactobacillus strain provided herein prevents adhesion of a LAP-expressing pathogenic bacteria provided herein by 71-80%. In another embodiment, the recombinant probiotic Lactobacillus strain provided herein prevents adhesion of a LAP-expressing pathogenic bacteria provided herein by 81-90%. In another embodiment, the recombinant probiotic Lactobacillus strain provided herein prevents adhesion of a LAP-expressing pathogenic bacteria provided herein by 91-100%.

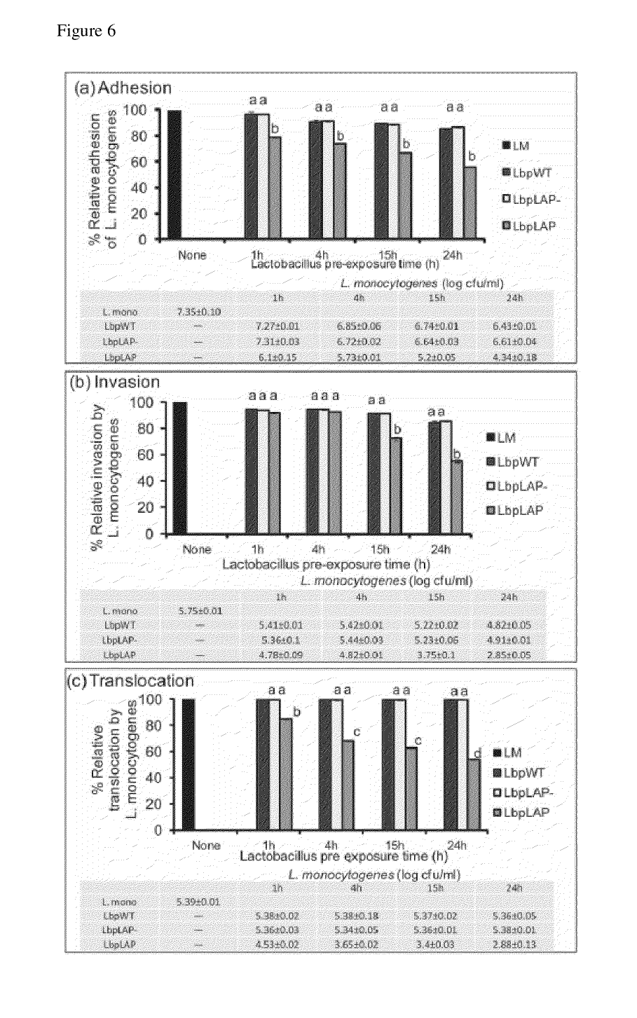

In one embodiment, the recombinant probiotic bacteria provided herein reduce invasion of pathogenic bacteria to the host cell. In another embodiment, the recombinant probiotic Lactobacilli provided herein prevents invasion of L. monocytogenes to the host by 44% (see FIG. 6b). In another embodiment, the recombinant probiotic Lactobacillus strain provided herein prevents invasion of a LAP-expressing pathogenic bacteria provided herein by 5-10%. In another embodiment, the recombinant probiotic Lactobacillus strain provided herein prevents invasion of a LAP-expressing pathogenic bacteria provided herein by 11-20%. In another embodiment, the recombinant probiotic Lactobacillus strain provided herein prevents invasion of a LAP-expressing pathogenic bacteria provided herein by 21-30%. In another embodiment, the recombinant probiotic Lactobacillus strain provided herein prevents invasion of a LAP-expressing pathogenic bacteria provided herein by 31-40%. In another embodiment, the recombinant probiotic Lactobacillus strain provided herein prevents invasion of a LAP-expressing pathogenic bacteria provided herein by 41-50%. In another embodiment, the recombinant probiotic Lactobacillus strain provided herein prevents invasion of a LAP-expressing pathogenic bacteria provided herein by 51-60%. In another embodiment, the recombinant probiotic Lactobacillus strain provided herein prevents invasion of a LAP-expressing pathogenic bacteria provided herein by 61-70%. In another embodiment, the recombinant probiotic Lactobacillus strain provided herein prevents invasion of a LAP-expressing pathogenic bacteria provided herein by 71-80%. In another embodiment, the recombinant probiotic Lactobacillus strain provided herein prevents invasion of a LAP-expressing pathogenic bacteria provided herein by 81-90%. In another embodiment, the recombinant probiotic Lactobacillus strain provided herein prevents invasion of a LAP-expressing pathogenic bacteria provided herein by 91-100%.

In one embodiment, the recombinant probiotic bacteria provided herein reduce translocation of a pathogenic to the host cell. In another embodiment, the recombinant probiotic Lactobacilli provided herein prevents translocation of L. monocytogenes to the host by 44% (see FIG. 6c). In another embodiment, the recombinant probiotic Lactobacillus strain provided herein prevents translocation of a LAP-expressing pathogenic bacteria provided herein by 5-10%. In another embodiment, the recombinant probiotic Lactobacillus strain provided herein prevents translocation of a LAP-expressing pathogenic bacteria provided herein by 11-20%. In another embodiment, the recombinant probiotic Lactobacillus strain provided herein prevents translocation of a LAP-expressing pathogenic bacteria provided herein by 21-30%. In another embodiment, the recombinant probiotic Lactobacillus strain provided herein prevents translocation of a LAP-expressing pathogenic bacteria provided herein by 31-40%. In another embodiment, the recombinant probiotic Lactobacillus strain provided herein prevents translocation of a LAP-expressing pathogenic bacteria provided herein by 41-50%.

In another embodiment, the recombinant probiotic Lactobacillus strain provided herein reduces adhesion of a LAP-expressing pathogenic bacterium by 20-30%. In another embodiment, the recombinant probiotic Lactobacillus strain provided herein reduces adhesion of a LAP-expressing pathogenic bacteria provided herein by 31-40%. In another embodiment, the recombinant probiotic Lactobacillus strain provided herein reduces adhesion of a LAP-expressing pathogenic bacterium by 41-50%. In another embodiment, the recombinant probiotic Lactobacillus strain provided herein reduces adhesion of a LAP-expressing pathogenic bacteria provided herein by 51-60%. In another embodiment, the recombinant probiotic Lactobacillus strain provided herein reduces adhesion of a LAP-expressing pathogenic bacteria provided herein by 61-70%. In another embodiment, the recombinant probiotic Lactobacillus strain provided herein reduces adhesion of a LAP-expressing pathogenic bacteria provided herein by 71-80%. In another embodiment, the recombinant probiotic Lactobacillus strain provided herein reduces adhesion of a LAP-expressing pathogenic bacteria provided herein by 81-90%. In another embodiment, the recombinant probiotic Lactobacillus strain provided herein reduces adhesion of a LAP-expressing pathogenic bacteria provided herein by 91-100%.

In one embodiment, the recombinant probiotic bacteria provided herein reduce invasion of pathogenic bacteria to the host cell. In another embodiment, the recombinant probiotic Lactobacilli provided herein reduces invasion of L. monocytogenes to the host by 44% (see FIG. 6b). In another embodiment, the recombinant probiotic Lactobacillus strain provided herein reduces invasion of a LAP-expressing pathogenic bacteria provided herein by 5-10%. In another embodiment, the recombinant probiotic Lactobacillus strain provided herein reduces invasion of a LAP-expressing pathogenic bacteria provided herein by 11-20%. In another embodiment, the recombinant probiotic Lactobacillus strain provided herein reduces invasion of a LAP-expressing pathogenic bacteria provided herein by 21-30%. In another embodiment, the recombinant probiotic Lactobacillus strain provided herein reduces invasion of a LAP-expressing pathogenic bacteria provided herein by 31-40%. In another embodiment, the recombinant probiotic Lactobacillus strain provided herein reduces invasion of a LAP-expressing pathogenic bacteria provided herein by 41-50%. In another embodiment, the recombinant probiotic Lactobacillus strain provided herein reduces invasion of a LAP-expressing pathogenic bacteria provided herein by 51-60%. In another embodiment, the recombinant probiotic Lactobacillus strain provided herein reduces invasion of a LAP-expressing pathogenic bacteria provided herein by 61-70%. In another embodiment, the recombinant probiotic Lactobacillus strain provided herein reduces invasion of a LAP-expressing pathogenic bacteria provided herein by 71-80%. In another embodiment, the recombinant probiotic Lactobacillus strain provided herein reduces invasion of a LAP-expressing pathogenic bacteria provided herein by 81-90%. In another embodiment, the recombinant probiotic Lactobacillus strain provided herein reduces invasion of a LAP-expressing pathogenic bacteria provided herein by 91-100%.

In one embodiment, the recombinant probiotic bacteria provided herein reduce translocation of a pathogenic bacterium to the host cell. In another embodiment, the recombinant probiotic Lactobacilli provided herein reduces translocation of L. monocytogenes to the host by 44% (see FIG. 6c). In another embodiment, the recombinant probiotic Lactobacillus strain provided herein reduces translocation of a LAP-expressing pathogenic bacteria provided herein by 5-10%. In another embodiment, the recombinant probiotic Lactobacillus strain provided herein reduces translocation of a LAP-expressing pathogenic bacteria provided herein by 11-20%. In another embodiment, the recombinant probiotic Lactobacillus strain provided herein reduces translocation of a LAP-expressing pathogenic bacteria provided herein by 21-30%. In another embodiment, the recombinant probiotic Lactobacillus strain provided herein reduces translocation of a LAP-expressing pathogenic bacteria provided herein by 31-40%. In another embodiment, the recombinant probiotic Lactobacillus strain provided herein reduces translocation of a LAP-expressing pathogenic bacteria provided herein by 41-50%.

In one embodiment, the recombinant Lactobacillus strain provided herein reduces L. monocytogenes-mediated cytotoxicity by 99.8% after 1 h of pre-exposure (prior to Listeria infection), 88.8% after 4 h, 80% after 15 h, and 79% after 24 h of pre-exposure, whereas LbpWT demonstrated no discernable protective effects (see Table 6).

In one embodiment, the recombinant probiotic bacteria strain effectively excludes L. monocytogenes from adhering to host cells when added before (inhibition of adhesion) or simultaneously (competitive adhesion).

It is to be understood that the methods of the present invention may be used to prevent or treat any pathogenic bacterial infection which uses LAP to adhere to and invade a host mammalian cell.

It is also to be understood that the probiotic bacteria provided herein can be used to prevent LAP-expressing pathogenic bacterial infections in high-risk individuals. In one embodiment, a high-risk individual includes but is not limited to, an immunocompromised human, a pregnant woman, a child, a human undergoing chemotherapy, a human receiving immunosuppressive drugs to treat cancer or to prevent a transplant rejection, or an elderly human.

In another embodiment, the methods of the present invention comprise the step of administering recombinant probiotic bacteria, in any form or embodiment as described herein. In one embodiment, the methods of the present invention consist of the step of administering recombinant probiotic bacteria of the present invention, in any form or embodiment as described herein. In another embodiment, the methods of the present invention consist essentially of the step of administering recombinant probiotic bacteria of the present invention, in any form or embodiment as described herein. In one embodiment, the term “comprise” refers to the inclusion of the step of administering a recombinant probiotic bacteria in the methods, as well as inclusion of other methods or treatments that may be known in the art. In another embodiment, the term “consisting essentially of” refers to a methods, whose functional component is the administration of recombinant probiotic bacteria, however, other steps of the methods may be included that are not involved directly in the therapeutic effect of the methods and may, for example, refer to steps which facilitate the effect of the administration of recombinant probiotic bacteria. In one embodiment, the term “consisting” refers to a method of administering recombinant probiotic bacteria with no additional steps.

In another embodiment, the recombinant probiotic bacterial strain provided herein is administered to a subject via the oral or intranasal route. In another embodiment, taking the compositions provided herein via the oral or intranasal route induces mucosal immunity against infectious agents present in the subject.

In one embodiment, the LAP-expressing recombinant probiotic bacteria provided herein have a two-fold advantage: direct antimicrobial effect against a target pathogen though the expression of the foreign gene and indirect general health benefits through the consumption of probiotics, which include, but are not limited to providing health maintenance by preventing or alleviating enteric infection, allergic diseases, and chronic inflammatory diseases such as inflammatory bowel disease, Crohn's disease, and ulcerative colitis. These beneficiary health effects may be achieved, in another embodiment, by increased defensin production, induction of anti-inflammatory responses, suppression of pro-inflammatory cytokines (i.e., tumor necrosis factor-alpha, interleukin-8 (IL-8), IL-6, increased production of short-chain fatty acids (butyrate) during fermentation, and improved epithelial tight junction barrier function.

In one embodiment, LAP-expressing probiotics are taken orally as a dietary supplement in a liquid or capsule form in a regular basis by this population during the period of need. In another embodiment, methods of administering are well known in the art and include, but are not limited to, oral administration, parenteral administration, intravenous (IV) administration, intranasal administration, or intraperitoneal (IP) administration.

Acceptable daily intake of probiotics is 35 g/day for a person weighing 70 kg, which is much higher than what is normally consumed and suggests very low risk of infection. Various embodiments of dosage ranges for probiotic bacteria are contemplated by this invention. In one embodiment, the dosage is 36-45 g/day for a person weighing 70 kg. In another embodiment, the dosage is 25-34 g/day for a person weighing 70 kg. In another embodiment, the dosage is 46-55 g/day for a person weighing 70 kg. It is to be understood that a skilled artisan, for example, a clinician can readily determine the appropriate dosage to be administered to a patient according to the patient's weight and health status. Alternatively, the skilled artisan can empirically determine the appropriate dosage based on the reduction of symptoms in a patient having a pathogenic infection. Each possibility represents a separate embodiment of the present invention.

In one embodiment, the term “pharmaceutically acceptable carrier” or “carrier” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of wetting agents. Compositions comprising such carriers are formulated by well known conventional methods (see, for example, Remington's Pharmaceutical Sciences, Chapter 43, 14th Ed. or latest edition, Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutical compositions containing the compositions of the present invention are, in another embodiment, administered to a subject by any method known to a person skilled in the art, such as parenterally, transmucosally, transdermally, intramuscularly, intravenously, intra-dermally, subcutaneously, intra-peritonealy, or intra-ventricularly.

In another embodiment of the methods and compositions provided herein, the compositions, i.e., the probiotic bacteria, are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelatin capsule.

In another embodiment, suitable liquid formulations include solutions, suspensions, dispersions, emulsions, oils and the like.

Other forms suitable for oral administration include liquid form preparations such as emulsions, syrups, elixirs, aqueous solutions, aqueous suspensions, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by mixing the inactivated probiotic bacteria with water and adding suitable colorants, flavors, stabilizing and thickening agents. Aqueous suspensions can be prepared by dispersing the inactivated probiotic bacteria in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

Exemplary pharmaceutically carriers include sterile aqueous or non-aqueous solutions, suspensions, and emulsions. Examples of non-aqueous solvents are propylene glycol, polyethylene glycol, vegetable or seed oils such as olive oil, and injectable organic esters such as ethyl oleate. Aqueous carriers include water, alcoholic/aqueous solutions, emulsions or suspensions, including saline and buffered media. A composition of inactivated probiotic bacteria may also be lyophilized using means well known in the art, for subsequent reconstitution and use according to the invention. Also of interest are formulations for liposomal delivery, and formulations comprising of encapsulated or microencapsulated inactivated probiotic bacteria.

The formulations of the present invention may also include known antioxidants, buffering agents, and other agents such as coloring agents, flavorings, vitamins or minerals. For example, a subject formulation may also contain one or more of the following minerals: calcium citrate (15-350 mg); potassium gluconate (5-150 mg); magnesium citrate (5-15 mg); and chromium picollinate (5-200 ng). In addition, a variety of salts may be utilized, including calcium citrate, potassium gluconate, magnesium citrate and chromium picollinate. Thickening agents may be added to the compositions such as polyvinylpyrrolidone, polyethylene glycol or carboxymethylcellulose. Exemplary additional components of a subject formulation include assorted colorings or flavorings, vitamins, fiber, milk, fruit juices, enzymes and other nutrients. Exemplary sources of fiber include any of a variety of sources of fiber including, but not limited to: psyllium, rice bran, oat bran, corn bran, wheat bran, fruit fiber and the like. Dietary or supplementary enzymes such as lactase, amylase, glucanase, catalase, and the like can also be included. Chemicals used in the present compositions can be obtained from a variety of commercial sources, including, e.g., Spectrum Quality Products, Inc (Gardena, Calif.), Sigma Chemicals (St. Louis, Mo.), Seltzer Chemicals, Inc., (Carlsbad, Calif.) and Jarchem Industries, Inc., (Newark, N.J.).

A subject formulation may also include a variety of carriers and/or binders. An exemplary carrier is micro-crystalline cellulose (MCC) added in an amount sufficient to complete dosage total weight. Carriers can be solid-based dry materials for formulations in tablet, capsule or powdered form, and can be liquid or gel-based materials for formulations in liquid or gel forms, which forms depend, in part, upon the routes of administration.

Typical carriers for dry formulations include, but are not limited to: trehalose, malto-dextrin, rice flour, micro-crystalline cellulose (MCC) magnesium sterate, inositol, fructo-oligosaccharide (FOS), gluco-oligosaccharide (GOS), dextrose, sucrose, and like carriers. Where the composition is dry and includes evaporated oils that produce a tendency for the composition to cake (adherence of the component spores, salts, powders and oils), dry fillers which distribute the components and prevent caking are included. Exemplary anti-caking agents include MCC, talc, diatomaceous earth, amorphous silica and the like, and are typically added in an amount of from approximately 1% to 95% by weight. It should also be noted that dry formulations which are subsequently rehydrated (e.g., liquid formula) or given in the dry state (e.g., chewable wafers, pellets, capsules, or tablets) can be used instead of initially hydrated formulations. Dry formulations (e.g., powders) may be added to supplement commercially available foods (e.g., liquid formulas, strained foods, or drinking water supplies). Similarly, the specific type of formulation depends upon the route of administration.

Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Generally, water-based carriers possess a neutral pH value (e.g., pH 7.0±1.0 or 0.5 pH units). The compositions may also include natural or synthetic flavorings and food-quality coloring agents, all of which must be compatible with maintaining viability of the lactic acid-producing microorganism. Well-known thickening agents may also be added to the compositions such as corn starch, guar gum, xanthan gum, and the like.

In another embodiment, oral dosage forms of the present invention prepared by any known or otherwise effective techniques known in the art that are suitable to provide final product forms of capsule, chewable tablet, swallowable tablet/pill, buccal tablet, coated tablet, troche, powder, lozenge, soft chew, solution, suspension, spray, extract, tincture, oil, decoction, infusion, syrup, elixir, wafer, food product such as acidified milk, yogurt, milk powder, tea, juice, beverage, confection (which includes candies and chocolates), chewable bar, cookie, wafer, cracker, cereal, treat, and combinations thereof, for oral ingestion and absorption to prevent or treat gastrointestinal diseases, conditions, symptoms and/or provide health benefits.

In one embodiment of the present invention, “nucleic acids” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

Any of a variety of expression vectors known to those of ordinary skill in the art may be employed to express recombinant polypeptides described herein. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are Lactobacilli strains provided herein.

The skilled artisan, when equipped with the present disclosure and the methods provided herein, will readily understand that different transcriptional promoters, terminators, carrier vectors or specific gene sequences (e.g. those in commercially available cloning vectors) can be used successfully in methods and compositions of the present invention. As is contemplated in the present invention, these functionalities are provided in, for example, the commercially available vectors known as the pUC series. In another embodiment, non-essential DNA sequences (e.g. antibiotic resistance genes) are removed. Each possibility represents a separate embodiment of the present invention. In another embodiment, a commercially available plasmid is used in the present invention. Such plasmids are available from a variety of sources, for example, Invitrogen (La Jolla, Calif.), Stratagene (La Jolla, Calif.), Clontech (Palo Alto, Calif.), or can be constructed using methods well known in the art.

Another embodiment is a plasmid such as one provided in Table 1, (see Examples below), which is a prokaryotic expression vector with a prokaryotic origin of replication and promoter/regulatory elements to facilitate expression in a prokaryotic organism. In another to embodiment, extraneous nucleotide sequences are removed to decrease the size of the plasmid and increase the size of the cassette that can be placed therein. Such methods are well known in the art, and are described in, for example, Sambrook et al. (1989, Molecular Cloning: A Laboratory Manual, Cold Spring Harbor Laboratory Press, New York) and Ausubei et al. (1997, Current Protocols in Molecular Biology, Green & Wiley, New York).

In one embodiment, antibiotic resistance genes are used in the conventional selection and cloning processes commonly employed in molecular biology and vaccine preparation. Antibiotic resistance genes contemplated in the present invention include, but are not limited to, gene products that confer resistance to ampicillin, penicillin, methicillin, streptomycin, erythromycin, kanamycin, tetracycline, cloramphenicol (CAT), neomycin, hygromycin, gentamicin and others well known in the art. Each gene represents a separate embodiment of the present invention.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, D.C.; Miller, 1992, A Short Course in Bacterial Genetics, Cold Spring Harbor Laboratory Press, Cold Spring Harbor, N.Y.) In another embodiment, the Listeria vaccine strain of the present invention is transformed by electroporation. Each method represents a separate embodiment of the present invention.

In another embodiment, conjugation is used to introduce genetic material and/or plasmids into bacteria. Methods for conjugation are well known in the art, and are described, for example, in Nikodinovic J et al. (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102 (35):12554-9). Each method represents a separate embodiment of the present invention.

“Transforming,” in one embodiment, is used identically with the term “transfecting,” and refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the present invention.

Plasmids and other expression vectors useful in the present invention are described elsewhere herein, and can include such features as a promoter/regulatory sequence, an origin of replication for gram negative and gram positive bacteria, and an isolated nucleic acid encoding a heterologous protein. Further, an isolated nucleic acid encoding a heterologous protein such as LAP, provided herein, will have a promoter suitable for driving expression of such an isolated nucleic acid. Promoters useful for driving expression in a bacterial system are well known in the art, and include bacteriophage lambda, the bla promoter of the beta-lactamase gene of pBR322, and the CAT promoter of the chloramphenicol acetyl transferase gene of pBR325. Further examples of prokaryotic promoters include the major right and left promoters of 5 bacteriophage lambda (PL and PR), the trp, recA, lacZ, lad, and gal promoters of E. coli, the alpha-amylase (Ulmanen et al, 1985. J. Bacteriol. 162:176-182) and the S28-specific promoters of B. subtilis (Gilman et al, 1984 Gene 32:11-20), the promoters of the bacteriophages of Bacillus (Gryczan, 1982, In: The Molecular Biology of the Bacilli, Academic Press, Inc., New York), and Streptomyces promoters (Ward et al, 1986, Mol. Gen. Genet. 203:468-478). Additional prokaryotic promoters contemplated in the present invention are reviewed in, for example, Glick (1987, J. Ind. Microbiol. 1:277-282); Cenatiempo, (1986, Biochimie, 68:505-516); and Gottesman, (1984, Ann Rev. Genet. 18:415-442).

In another embodiment, a plasmid of the methods and compositions provided herein comprises a gene encoding a heterologous protein. In another embodiment, the heterologous protein is LAP.

In another embodiment, subsequences are cloned and the appropriate subsequences cleaved using appropriate restriction enzymes. The fragments are then, in another embodiment, ligated to produce the desired DNA sequence. In another embodiment, DNA encoding the antigen is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The gene coding the antigen is ligated into a plasmid. Each method represents a separate embodiment of the present invention.

The recombinant proteins of the present invention are synthesized, in another embodiment, using recombinant DNA methodology, for example, cloning and restriction of appropriate sequences or direct chemical synthesis by methods such as the phosphotriester method of Narang et al. (1979, Meth. Enzymol. 68: 90-99); the phosphodiester method of Brown et al. (1979, Meth. Enzymol 68: 109-151); the diethylphosphoramidite method of Beaucage et al. (1981, Tetra. Lett., 22: 15 1859-1862); and the solid support method of U.S. Pat. No. 4,458,066.

In another embodiment, chemical synthesis is used to produce a single stranded oligonucleotide. This single stranded oligonucleotide is converted, in various embodiments, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences can be obtained by the ligation of shorter sequences. In another embodiment, subsequences are cloned and the appropriate subsequences cleaved using appropriate restriction enzymes. The fragments are then be ligated to produce the desired DNA sequence.

In another embodiment, DNA encoding the recombinant protein of the present invention is cloned using DNA amplification methods such as polymerase chain reaction (PCR). Thus, the gene for LAP is PCR amplified, using a sense primer comprising a suitable restriction site and an antisense primer comprising another restriction site, e.g. a non-identical restriction site to facilitate cloning. Insertion into a plasmid or vector produces a vector encoding LAP.

In one embodiment, protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In one embodiment, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

In one embodiment, the present invention provides a kit comprising: a) recombinant probiotic bacteria in capsule, liquid or any form provided herein, b) instructional materials for storage, c) instructional materials for use d) compliance aid. In another embodiment, the present invention provides a kit comprising a composition, tool, or instrument of the present invention.

The kits can also comprise maintenance doses of a maintenance probiotic to be administered for a maintenance time period; doses of an additional material to be administered for a maintenance time period; instructions for use of the kit; a compliance aid; and combinations thereof.

The kits of the present invention can also include one or more compliance aids for facilitating compliance and/of allowing the user to visually track progress. Non-limiting examples of a compliance aid which can be used to track progress include a diary, chart, fillable color coded chart, and tracking device, and combinations thereof. The compliance aid can be provided, contained, stores, and/or delivered in a variety of forms including, for example, paper, computer, personal digital assistant, telephone (including cellular phone and other communication devices). A compliance aid useful with the methods of the present invention is described in U.S. patent application Ser. No. 11/391,839.

The term “therapeutically effective dose” or “therapeutic effective amount” means a dose that produces the desired effect for which it is administered. The exact dose will be ascertainable by one skilled in the art using known techniques.

The term “subject” or “patient” refers a human at risk of having or actually having a pathogenic bacterial infection, for example, an L. monocytogenes or L. ivanovii infection, or any other LAP-expressing infectious disease. The term “subject” does not exclude an individual that is normal in all respects. Moreover, the terms “subject,” “host,” “patient,” and “individual” are used interchangeably herein to refer to any mammalian subject for whom diagnosis or therapy is desired, particularly humans. Other subjects may include cattle, sheep, goats, dogs, cats, guinea pigs, rabbits, rats, mice, horses, and so on.

The term “about” as used herein means in quantitative terms plus or minus 5%, or in another embodiment plus or minus 10%, or in another embodiment plus or minus 15%, or in another embodiment plus or minus 20%.

The following examples are presented in order to more fully illustrate the preferred embodiments of the invention. They should in no way be construed, however, as limiting the broad scope of the invention.

### EXAMPLES

### Materials and Methods

### Examples 1 to 7

### Bacterial Strains, Plasmids, and Growth Conditions

Bacterial strains and plasmids used in this study are listed in Table 1. All Listeria species were grown in brain heart infusion or Luria-Bertani broth (LB, 0.5% NaCl, 1% tryptone peptone, and 0.5% yeast extract) at 37° C. for 16 to 18 h. All lactic acid bacteria except Lactococcus lactis were cultured in deMan Rogosa Sharpe broth (MRS, Becton Dickinson) at 37° C. for 18-20 h. Lc. lactis strains were grown in M17 broth (Becton Dickinson). Lb. rhamnosus, Lb. paracasei, and Lb. gasseri were grown at 37° C. under anaerobic conditions. The lap deficient mutant L. monocytogenes strain KB208 was grown in BHI or LB with erythromycin (5 mg/mL) at 42° C. pLP401T was used for LAP expression in Lb. paracasei and was grown in appropriate media with ampicillin (50 mg/mL) for E. coli, and erythromycin (2 mg/mL) for Lb. paracasei. To induce expression of LAP in recombinant Lb. paracasei, the bacterium was grown in modified MRS (1% w/v proteose peptone, 0.5% w/v yeast extract, 0.2% w/v meat extract, 0.1% v/v Tween 80, 37 mM C₂H₃NaO₂, 0.8 mM MgSO₄, 0.24 mM MnSO₄, 8.8 mM C₆H₁₄N₂O₇ in 0.1 M potassium phosphate buffer, pH 7.0) supplemented with mannitol (1% w/v).

TABLE 1
Bacterial strains and plasmids.

| Bacteria/plasmids | Strains | Description |
|---|---|---|
| Listeria monocytogenes | F4244 | Wild type, serotype 4b |
| L. monocytogenes | KB208 | F4244, LAP deficient strain (EmR 5 mg/mL) |
| L. innocua | F4248 | Wild type |
| Lactobacillus acidophilus | NRRL B1910 | Wild type |
| Lb. casei | KCTC 3109 | Wild type |
| Lb. gasseri | ATCC19992 | Wild type |
| Lb. paracasei | DUP13076 | Wild type |
| Lb. paracasei | LAP+ | Lb. paracasei expressing LAP of |
| | (AKB901) | L. monocytogenes (EmR 2 mg/ml) |
| Lb. paracasei | LAP− | Lb. paracasei carrying control plasmid with no |
| | | insert (EmR 2 mg/ml) |
| Lb. plantarum | NCDO | Wild type |
| Lb. rhamnosus GG | ATCC53103 | Wild type |
| Pediococcus acidilactici | H | Pediocin AcH-producing strain, Wild type |
| Ped. acidilactici | RS2 | Pediocin RS2-producing strain; Wild type |
| Lactococcus lactis | ATCC 7962 | Wild type |
| Lac. lactis | ATCC 11454 | Nisin-producing strain; Wild type |
| Plasmids | | |
| pGEM-T easy | | Cloning vector (AmR 50 mg/mL) |
| pGEM-LAPLm | | pGEM-Teasy carrying lap of L. monocytogenes |
| pLP401T | | Expression vector for Lactobacillus, (AmR 50 |
| | | mg/mL and EmR 2 mg/mL) |
| pLP401-LAP | | pLP401 carrying lap of L. monocytogenes |

KCTC, Korean Type Culture Collection; ATCC, American Type Culture Collection; NCDO, National Collection of Dairy Organisms.

### Construction of LbpLAP

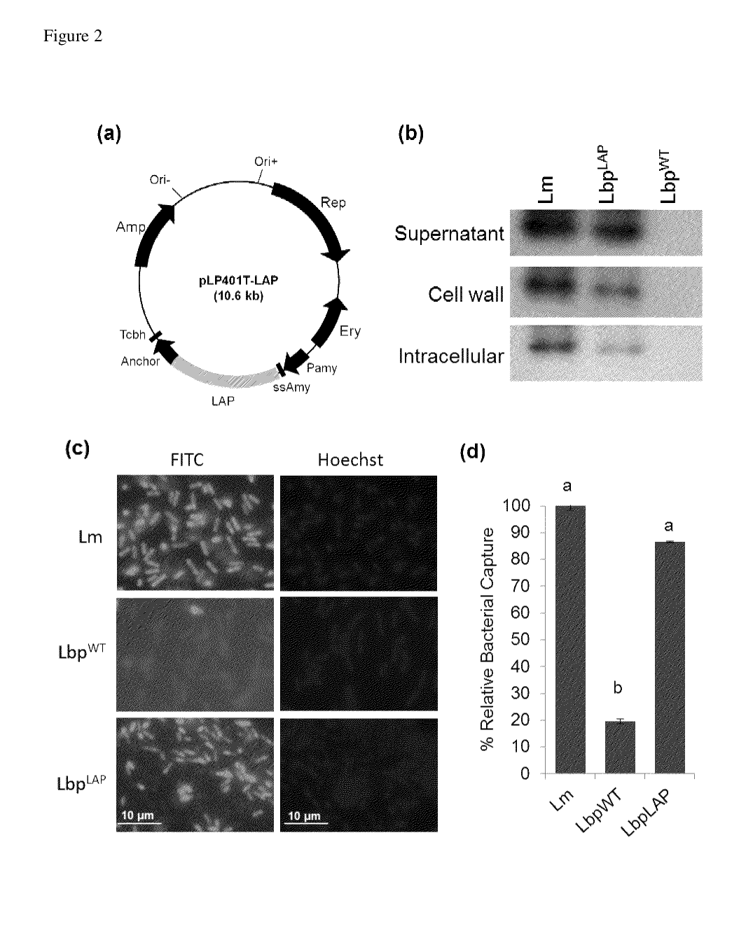

Genomic DNA from L. monocytogenes F4244 was purified and the lap gene was amplified from genomic DNA with polymerase chain reaction using primers LAPLmN-F 59-GACCATGGATGGCAATTAAAGAAAATG-39 (SEQ ID NO: 1) and LAPLmX-R59-GACTCGAGTCAAACACCTTTGTAAG-39 (SEQ ID NO: 2). The amplified DNA was cloned into pGEM-T Easy Vector and designated pGEMLAPLm. Lactobacilli expression vector, pLP401-T was used to express LAP in Lb. paracasei (48; FIG. 2a). This vector has been shown to be efficient for heterologous protein delivery by lactobacilli, owing to the presence of a secretion signal and the leader sequence of cell wall proteinase (prtP) from Lb. casei. This gene sequence codes for the secretion and surface association of heterologous proteins to the peptidoglycan. The plasmid was digested with NcoI and XhoI, inserted into expression vector pLP401T, and designated pLP401T-LAP. To remove the terminator, which stabilizes the plasmid in E. coli, pLP401T-LAP was digested with NotI and pLP401T-LAP was obtained via self-ligation. Self-ligated pLP401T-LAP was transformed into Lb. paracasei by electroporation. Competent Lb. paracasei cells were prepared with incubation of 2% culture in fresh MRS broth containing 0.5% sucrose and 0.5% glycine at 37° C. until OD600 reached to 0.5, 0.8. The cells were harvested (3,900 g for 5 mM at 4° C.), washed twice with washing buffer (0.5 M sucrose, 10% glycerol) and collected. Then the cells were resuspended in the same washing buffer and stored at −80° C. For electroporation, 50 ml of competent cells mixed with 0.5 mg of purified plasmid DNA in an ice cold cuvette with a 2-mm electrode gap. The electric pulse was delivered by the Gene Pulser electroporation system using the following parameter settings: 1.5 kV, 200V and 25 mF. After electroporation, competent cells were recovered in 1 ml of MRS containing 0.5 M sucrose, 20 mM MgCl₂, 2 mM CaCl₂ at 37° C. for 2 h in water bath. Transformants were selected using MRS agar containing 2 mg/mL of erythromycin. Similarly, another recombinant strain was generated carrying the pLP401-T plasmid with no LAP insert to be used as a vector control (Lbp^{LAP-}). Identity of recombinant and WT Lb. paracasei strains were confirmed by ribotyping using an automated RiboPrinterH. Protein expression in recombinant strains was confirmed with Western blot analysis.

Analysis of LAP Expression by Lb. paracasei

LAP expression in SN, CW, and intracellular fractions was analyzed. SN was collected from centrifuged culture (7,000 g for 10 mM at 4° C.) and the pellet was retained for preparation of CW and intracellular proteins. The SN was filtered (0.22-mm filter), precipitated to with 10% trichloroacetic acid for 40 mM on ice, and centrifuged (14,000 g at 4° C. for 10 mM) The pellet was resuspended in ice-cold acetone and centrifuged. The remaining acetone was evaporated, and the pellet was resuspended in alkaline rehydration buffer (100 mM Tris-base, 3% SDS, 3 mM dithiothreitol, pH 11), boiled for 5 mM, and stored at −20° C. For the CW protein fraction, the pellet was resuspended in 5 M LiCl with 5 mM EDTA and incubated for 30 mM in a water bath at 37° C. The suspension was centrifuged (13,000 g at 4° C. for 5 min) and the SN was filtered (0.45-mm filter). The sample was dialyzed using ultrapure water supplemented with 5 mM EDTA and stored at −20° C.

The pellet from the CW protein preparation was used for intracellular protein isolation. It was resuspended in the sample solvent (5% SDS, 0.5% b-mercaptoethanol, 1.5% Tris, pH 7.0) and sonicated on ice for 5-7 cycles of 15 sec each using a Sonifier 150D. The sample was centrifuged and the SN was collected and stored at − (negative) 20° C. SN and CW protein preparations were also tested with a PepC assay to rule out contamination with intracellular or membrane proteins.

Proteins were quantified using the bicinchoninic acid method and equivalent amounts of protein (40 μg of each fraction) were separated using SDS polyacrylamide gel electrophoresis (7.5% acrylamide) gel. The proteins were transferred to an Immobilon-P membrane and immunoprobed with anti-LAP antibody MAb-H7 (1.0 mg/mL) and horseradish peroxidase-coupled anti-mouse antibody (0.2 mg/mL). The membranes were developed with an enhanced chemiluminescence kit.

LAP expression in recombinant probiotics was also determined by reacting 18-h grown bacterial cells first with MAb-H7 for 1 h followed by FITC-labeled anti-mouse monovalent secondary Fab fragment (diluted 1:250 in phosphate-buffered saline [PBS]; Jackson Immuno Research) for 1 h and counterstained with Hoechst dye (0.5 mg/mL in PBS; Invitrogen) for nucleus staining Cells were washed between antibody treatments with PBS containing 1% bovine serum albumen and examined under a fluorescence microscope equipped with SPOT software.

Analysis of Recombinant Probiotic Interaction with Hsp60-Coated Paramagnetic Beads

A magnetic bead capture method was used to analyze the interactions of surface-associated LAP on the recombinant probiotic with human Hsp60. Paramagnetic beads (Streptavidin C1 beads; average diameter, 1.0 μm) were coated with biotinylated Hsp60 as described elsewhere.

Briefly, PBS-washed, overnight-grown bacterial cells (250 mL) were mixed gently with Hsp60-coated beads (20 mL) and incubated at 25° C. for 1 h on a vortex mixer. The beads were removed using a magnetic particle concentrator (MPC-S) and washed three times with PBS (20 mM, pH 7.0) and once with PBS containing 0.5% Tween 20. Captured bacteria were mechanically separated from the beads by vigorous vortexing; lactobacilli were quantified by plating on MRS agar and Listeria on modified oxford (MOX) agar plates after incubation at 37° C. for 24-48 h.

### Adhesion Assays

Human colon carcinoma cell line, Caco-2 (HTB37; American Type Culture Collection) was cultured in Dulbecco's modified Eagle's medium supplemented with 10% fetal bovine serum (D10F). Passages of 20-35 were used for the experiments, and the cells were grown in 12- and 24-well plates at 37° C. in the presence of 7% CO₂ in a cell culture incubator for 10-12 days or until monolayers formed with no further visible differentiation.

The adhesion profiles of bacteria (10⁶ cfu/well) to Caco-2 cells (10⁵ cells/well) with multiplicity of exposure (MOE) of 10:1 were analyzed using adhesion assays. Adhered LAB was enumerated on MRS and Listeria on MOX agar plates. Additionally, bacterial adhesion to cell monolayers grown on glass coverslips was done by Giemsa staining followed by microscopic examination to visualize bacterial attachment qualitatively. To verify LAP-mediated binding, bacterial cells were also pretreated with anti-LAP antibody before use in the adhesion experiment. As an immunoglobulin G isotype control, MAb EM-7G1 that reacts with a 66-kDa protein in L. monocytogenes was used.

### Invasion Assay

Invasion of bacteria was analyzed. Bacteria were added to Caco-2 cells at an MOE of 10:1 and incubated for 1 h. The monolayers were washed with D10F, and an additional 1 h of incubation in D10F. containing 50 μg/mL gentamicin followed. The cells were lysed with 0.1% Triton X-100 and plated for enumeration of internalized bacteria.

### Transepithelial Translocation Assay

Transepithelial bacterial translocation assay was performed. Briefly, Caco-2 cells were grown on transwell filter inserts (4-mm pore filter) for 10-12 days to reach confluence. Bacteria were added to the apical well of the insert and incubated for 2 h. Liquid from the basal well was removed, serially diluted if needed, and distributed onto plates for enumeration. TEER of Caco-2 cells before and after treatment was measured using a Millicell ERS system.

Competitive Exclusion of L. monocytogenes by LAB

Strains Competitive exclusion was determined using competitive adhesion, inhibition of adhesion, and displacement experiments. A ratio of 10:1 of L. monocytogenes or LAB strains to Caco-2 cells was used. (i) Competitive adhesion: L. monocytogenes and LAB strains were added simultaneously to Caco-2 cells and incubated for 1 h. To remove unbound bacteria, the cells were washed four times with Cell-PBS (137 mM NaCl, 5.4 mM KCl, 3.5 mM Na₂HPO₄, 4.4 mM NaH₂PO₄, 11 mM glucose, pH7.2). Adherent bacteria were released by treatment with 0.1% Triton-X 100 in Cell-PBS and plated onto MOX for L. monocytogenes and MRS agar for LAB strains. (ii) Inhibition of adhesion: LAB strains were added to wells containing Caco-2 cells and incubated for 1 h. Unbound bacteria were removed by washing with D 10F as above, and L. monocytogenes was then added and incubated for 1 h. The cells were then washed. Bound bacteria were released and plated as above. (iii) Displacement: L. monocytogenes were added to Caco-2 cells and incubated for 1 h. After washing with D10F, each LAB strain was added and incubated for 1 h. The cells were then washed. Bound bacteria were released and plated as above.

Inhibition of L. monocytogenes Adhesion, Invasion, and Translocation by LbpLAP

The ability of Lbp^{LAP} to inhibit L. monocytogenes adhesion, invasion, and translocation to Caco-2 cells was investigated. Lbp^{LAP} and Lbp^{WT} were added to each well and incubated for 1, 4, 15, or 24 h. Unbound bacteria were removed by washing with D10F, and L. monocytogenes was added (MOI; 10:1) and incubated for 1 h for inhibition of adhesion and invasion experiments and 2 h for inhibition of translocation experiments. The cells were then washed. Bound bacteria were released by Triton-X treatment and plated as above. As a vector control, the recombinant Lbp^{LAP}-strain was used to rule out the involvement of any plasmid encoded proteins in protection against L. monocytogenes infection.

### Epithelial Tight Junction Integrity Analysis

Tight junction permeability of Caco-2 monolayers in transwell filter inserts (4-mm pore size; Corning) pre-exposed to probiotics for 1, 4, 15 and 24 h and infected with L. monocytogenes for 2 h was assessed by monitoring Dextran FITC (Mr 3-5 kDa) permeability. MOE for all bacterial strains was 10:1. Dextran-FITC (1 mg/ml) was added to the transwell and incubated at 37° C. for 1 h. Samples from the apical and basolateral chambers were collected and read in a SpectraMax Gemini EM fluorescent plate reader. The data are expressed as percentages of the apical dextran recovered in the basal chamber.

### Cytotoxicity Assay and Fluorescence Microscopy

Caco-2 cell cytotoxicity was assessed using a lactate dehydrogenase cytotoxicity assay kit. Caco-2 cell viability was also assessed with live and dead staining of Caco-2 monolayers using a propidium iodide (PI; red, dead cell indicator) and acridine orange (AO, green, live cell indicator) mixture (PI; 100 μg/mL and AO; 20 μg/mL). Stained cells were washed in Cell-PBS, fixed in methanol, and examined under a fluorescence microscope equipped with SPOT software to using green (for AO) and red (for PI) filters.

### Statistical Analysis

All experiments were repeated at least three times independently, and each set of experiments was performed in duplicate or triplicate. Statistical comparisons were carried out using analysis of variance and Tukey's multiple comparisons of means at P<0.05 to determine significant differences.

### Materials and Methods

### Examples 8 to 14

### Bacterial Strains, Plasmids, and Growth Conditions

Bacterial strains and plasmids used in this study are listed in Table 2. All Listeria species were grown in brain heart infusion (BHI, Becton Dickinson, Sparks, Md.) or Luria-Bertani broth (LB, 0.5% NaCl, 1% tryptone peptone, and 0.5% yeast extract) at 37° C. for 16 to 18 h. Probiotic cultures were cultured in deManRogosaSharpe broth (MRS, Becton Dickinson) at 37° C. for 18-20 h. Lb. casei ATCC 344 wild type (LbcWT) (a gift from Mike Miller, University of Illinois, Urbana) was used as a host to express Listeria Adhesion Protein (LAP) from L. innocua and L. monocytogenes (see below). To recover this strain from fecal and intestinal samples during animal study, a vancomycin resistant strain of Lb. casei was selected by sequentially culturing the bacterium in increasing concentrations of vancomycin (300 μg/ml). Recombinant Lb. paracasei was grown at 37° C. The lap-deficient mutant L. monocytogenes strain KB208 was grown in BHI or LB with erythromycin (5 μg/mL) at 42° C.

TABLE 2
Bacterial strains and plasmids

| Bacteria/plasmids | Strains | Description | Source |
|---|---|---|---|
| Listeria monocytogenes | F4244 | Wild type, serotype 4b | Our collection |
| L. monocytogenes | KB208 | F4244, LAP deficient strain (Em^{R} 5 μg/mL) | Our laboratory |
| L. innocua | F4248 | Wild type | Our collection |
| L. monocytogenes | AKB308 | KB208, LAP deficient strain (Em^{R} 5 | Our laboratory |
| | | μg/mL) expressing LAP of L. innocua | |
| Lb. paracasei | DUP13076 | Wild type | Lactrys |
| | (AKB900) | | Biopharmaceuticals |
| | | | BV (Netherlands) |
| Lb. paracasei | LbpLAP^{Lm} | Lb. paracasei expressing LAP of | This study |
| | (AKB901) | L. monocytogenes (Em^{R} 2 μg/ml) | |
| Lb. paracasei | LbpLAP⁻ | Lb. paracasei carrying control plasmid with | This study |
| | (AKB902) | no insert (Em^{R} 2 μg/ml) | |
| Lb. casei | ATCC334 | WT | M. Miller, Univ of |
| | (AKB905) | | Illinois, Urbana |
| Lb. casei | LbcLAP^{Lm} | Lb. casei expressing LAP of | This study |
| | (AKB906) | L. monocytogenes (Em^{R} 2 μg/ml) | |
| Lb. casei | LbpLAP^{Linn} | Lb. paracasei expressing LAP of L. innocua | This study |
| | (AKB907) | (Em^{R} 2 μg/ml) | |
| Lb. casei | LbpLAP⁻ | Lb. casei carrying control plasmid with no | This study |
| | (AKB908) | insert (Em^{R} 2 μg/ml) | |
| Plasmids | | | |
| pGEM-T easy | | Cloning vector (Am^{R} 50 μg/mL) | Promega |
| pGEM-LAPLm | | pGEM-Teasy carrying lap of | This study |
| | | L. monocytogenes | |
| pMGS101 | | Listeria expression vector | Fujimoto & Ike |
| | | | (2001) |
| pMGS101-LAP^{Lin} | | pMGS101 carrying lap of L. innocua | This study |
| pLP401T | | Expression vector for Lactobacillus, | (Maassen et al. |
| | | (Am^{R} 50 μg/mL and Em^{R} 2 μg/mL) | 1999) |
| pLP401-LAP^{Lm} | | pLP401 carrying lap of L. monocytogenes | This study |
| pLP401-LAP^{Lin} | | pLP401 carrying lap of L. innocua | This study |

ATCC, American Type Culture Collection; NCDO.

Generation of Recombinant Probiotic Lactobacilli Expressing LAP from Nonpathogenic Listeria innocua and L. monocytogenes

The entire lap gene (2.6 kb) from L. innocua was amplified by PCR and inserted into pLP401T and electro-transformed into Lactobacillus casei ATCC334 designated LbcLAP^{Linn} (Lb. casei AKB907). Likewise, lap gene from L. monocytogenes was expressed in Lb. casei designated LbcLAP^{Lm} (AKB906). The recombinant strains were maintained in MRS broth containing erythromycin (2 μg/ml). The entire lap gene from L. innocua was also cloned into pMGS101 and electrotransformed into LAP deficient L. monocytogenes mutant strain KB208 (Kim et al. 2006) and designated as LmKB208LAP^{Lin}. To induce expression of LAP, the recombinant Lb. casei strains, were grown in modified MRS (1% w/v proteose peptone, 0.5% w/v yeast extract, 0.2% w/v meat extract, 0.1% v/v Tween 80, 37 mM C₂H₃NaO₂, 0.8 mM MgSO₄, 0.24 mM MnSO₄, 8.8 mM C₆H₁₄N₂O₇ in 0.1 M potassium phosphate buffer, pH 7.0) supplemented with mannitol (1% w/v). LAP expression was verified by Western blotting, ELISA and immunofluorescence staining using monoclonal antibody to LAP (MAb-H7).

### Growth Characteristics of Recombinant Probiotics in Artificial Gastrointestinal Fluids

The survival of probiotics exposed sequentially to simulated gastrointestinal fluid (SGF, to simulate gastric phase) and simulated intestinal fluid (SIF-I and SIF-II, to simulate enteric phase 1 and enteric phase 2, respectively), over 6 h (2 h for each step) period was monitored. SGF contained pepsin (3 g/L) and lipase (0.9 mg/L) (Sigma-Aldrich), pH 1.2-1.5 (adjusted using 1N HCl) and both SIF-I and SIF-II contained bile (bovine bile; 10 g/L, Sigma-Aldrich) and porcine pancreatin (1 g/L; Sigma-Aldrich) but SIF-1 pH was 4.3-5.2 and SIF-II pH 6.7-7.5 (adjusted using alkaline solution; 150 ml of 1 N NaOH, 14 g of PO₄H₂Na.2H₂O and distilled water up to one 1 L). Overnight cultures of wild type or recombinant probiotics were washed and resuspended in SGF (100 ml) and incubated at 37° C., with agitation (150 rpm for 2 h) (gastric phase) and bacterial counts were monitored every 30 min for 2 h. The cells from SGF were pelleted down, and transferred sequentially into SIF-I and SIF-II, incubated each at 37° C. for 2 h tosimulate the initial and final phases of intestinal digestion. Probiotics counts were enumerated on MRS agar plates and the assay was repeated three times with duplicate samples. Viability was also verified by performing live and dead staining using cFDA-SE (carboxyfluoresceindiacetatesuccinimidyl ester, 50 μM) and PI (propidium iodide, 30 μM) as described (Lee et al., 2004). Levels of LAP expression in probiotic cultures during exposure to SGF and SIF was monitored by Immunofluorescence staining and Western blotting. Recombinant probiotics survival in water was also monitored to ensure probiotics remained viable during animal feeding for 24 h.

Inhibition of Listeria monocytogenes Adhesion, Invasion and Transepithelial Translocation by Recombinant Probiotics

The ability of wild type and recombinant probiotics (LbcLAP^{Linn} and LbcLAP^{Lm}) to inhibit L. monocytogenes adhesion, invasion, and translocation to Caco-2 cells was investigated as described elsewhere. Recombinant strains were added to each well and incubated for 24 h. Unbound bacteria were removed by washing with Dulbecco's modified Eagles' medium containing 10% fetal calf serum (D10F), and L. monocytogenes was added (MOI; 10:1) and incubated for 1 h to determine inhibition of adhesion and invasion. The cell monolayers were then washed three times and adherent bacteria were released by Triton-X treatment and plated. To determine intracellular bacteria, the cell monolayers were treated with gentamycin (50 μg/mL) for 1 h before Triton-X treatment. As a vector control, the recombinant LbcLAP⁻ strain was used to rule out the involvement of any plasmid encoded proteins in protection against L. monocytogenes infection.

Transepithelial bacterial translocation assay was performed as previously described (Burkholder and Bhunia 2010; Cruz et al. 1994). Briefly, Caco-2 cells were grown on transwell filter inserts (4-μm pore filter; Corning, Lowell, Mass.) for 10-12 days to reach confluence. Bacteria were added to the apical well of the insert and incubated for 2 h. Liquid from the basal well was removed, serially diluted if needed, and distributed onto plates for enumeration. TEER of Caco-2 cells before and after treatment was measured using a Millicell ERS system (Millipore, Billerica, Mass.).

### Mouse Bioassay

Female mice (A/J: 8-10 days of age; n=60) were purchased from Jackson laboratories to (Bar Harbor, Me.). The animal bioassay procedure was approved by the Purdue University Animal Care and Use Committee. Upon arrival, mice (2/cage) were housed in a cage that had a solid stainless divider to physically keep two separated. Shepherd's™ ALPHA-dri®, made from alpha cellulose was used for bedding. Animals were provided adlib feed (Rodent Diet 5001, LabDiet, Brentwood, Mo.) and water (Sterile deionized water), and acclimatized for 5 days before experiment. A cycle of 12 h artificial light and 12 h darkness was maintained. Relative humidity was 50-60% and temperature was 20-25° C.

Mice were randomly assigned to eight different groups. Probiotics were supplied in sterile deionized water at ˜9×10⁹ CFU/ml for 10 days and fresh preparation of probiotics were supplied daily. Control animals received only sterile drinking water. Probiotic colonization in the gut was monitored daily by analyzing fecal counts of probiotics daily. For challenge experiment, mice received oral gavage of L. monocytogenes F4244 (WT) at a concentration of 8×10⁸ CFU/mouse using a feeding tube (Popper) and control mice received PBS (Burkholder et al. 2009) Animals were observed periodically for clinical signs such as ruffled hair, movement and recumbence, and their feeding and drinking habits.

### Histopathology and Immunohistochemistry

Formalin-fixed and paraffin-embedded tissues were stained with Hematoxylin and Eosin by the Purdue University Histology and Phenotyping laboratory. Digital photomicrographs of tissue sections were taken with an Olympus BX microscope at the Histology Laboratory. Tissue sections were scored separately for inflammation and fibrosis in a blinded fashion by a pathologist (DM). Inflammation was scored based on the Wirtz scale: (0) no inflammation, (1) low level of inflammation with scattered infiltrating mononuclear cells, (2) moderate inflammation with multiple foci, (3) high level of inflammation with increased vascular density and marked wall thickening, (4) maximal severity of inflammation with transmural leukocyte infiltration and loss of goblet cells. Briefly, full thickness stained sections of mouse intestine were scanned using a Pathscan Enabler IV (Meyer Instruments, Houston, Tex.) slide scanner and digitized. The image was analyzed using the ImageScope software.

Paraffin-embedded intestinal sections fixed in formalin were pre-treated with heat-induced epitope retrieval solution and then blocked with Dako protein block according to manufacturer's instructions. Rabbit Anti-human CD3 (1:500) was used as the primary antibody followed by labeling with Dako labeled polymer. The stained slides were then scanned and analyzed as described previously.

### Results

### Example 1

### Lactobacilli Showed Highest Attachment to Caco-2 Cells

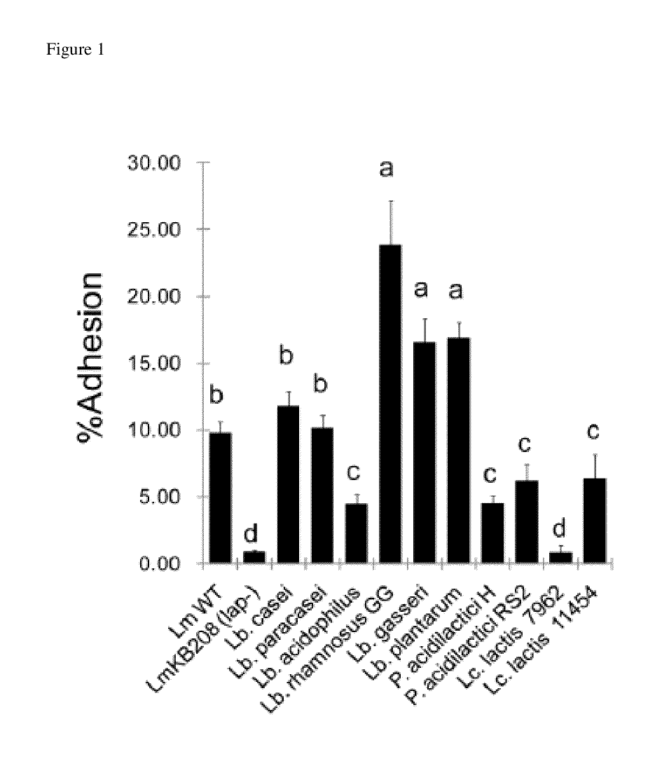

The ability to adhere to or colonize epithelial cells is an essential and prerequisite trait for probiotic bacteria. To select the most suitable candidate for genetic modification, the attachment profiles of several lactic acid bacteria (LAB) were screened, including some well-characterized probiotics (Lactobacillus spp.) and bacteriocin-producing strains (Pediococcus and Lactococcus) to Caco-2 cells (Table 1; FIG. 1). As reference controls, adhesion of L. monocytogenes F4244 (wild type [WT]) and the lap-deficient isogenic strain KB208 were also analyzed, showing about 9.78% and 0.84% adhesion, respectively. Attachment of LAB to Caco-2 cells varied from 0.78% to 23.8% with Lactobacillus rhamnosus showing the highest (23.8%) adhesion, followed by Lb. plantarum (16.9%), Lb. gasseri (16.6%), Lb. casei (11.8%), and Lb. paracasei (10.2%). Lb. acidophilus, Pediococcus, and Lactococcus attached to Caco-2 cells in significantly lower numbers than those of L. monocytogenes (P<0.0001). From this study, a representative strain of highly adherent Lb. rhamnosus, moderately high Lb. paracasei, and low adherent Lb. acidophilus were chosen for subsequent experiments.

### Example 2

### Wild Type Lactobacilli Do Not Reduce L. monocytogenes

### Infection in Caco-2 Cells.

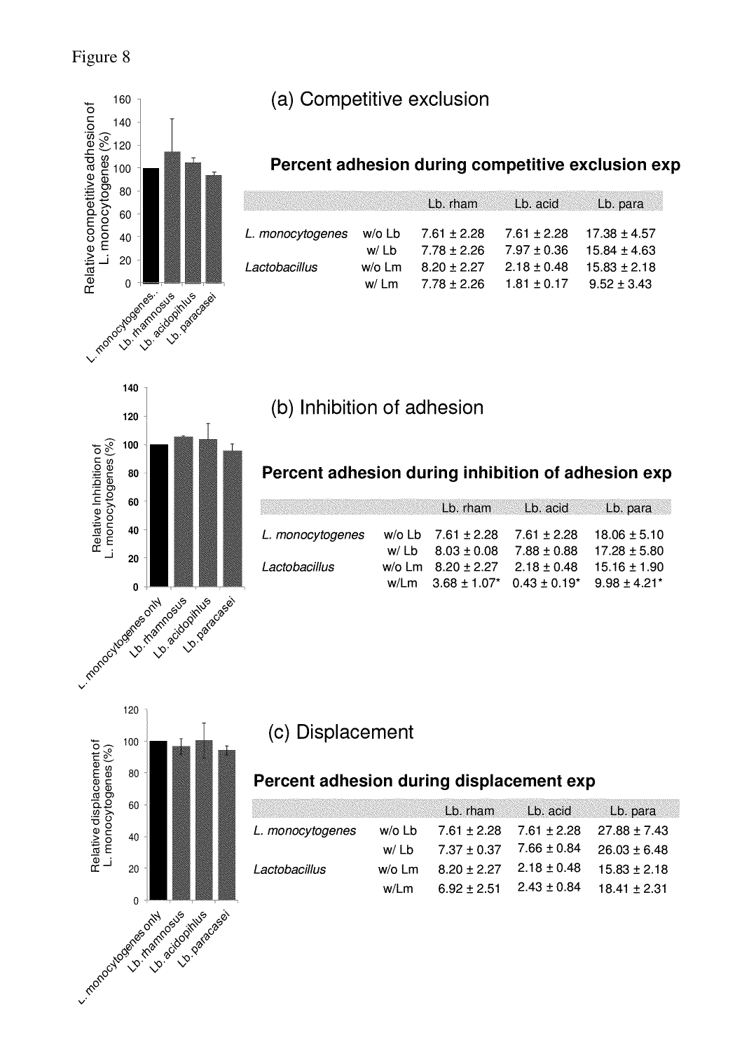

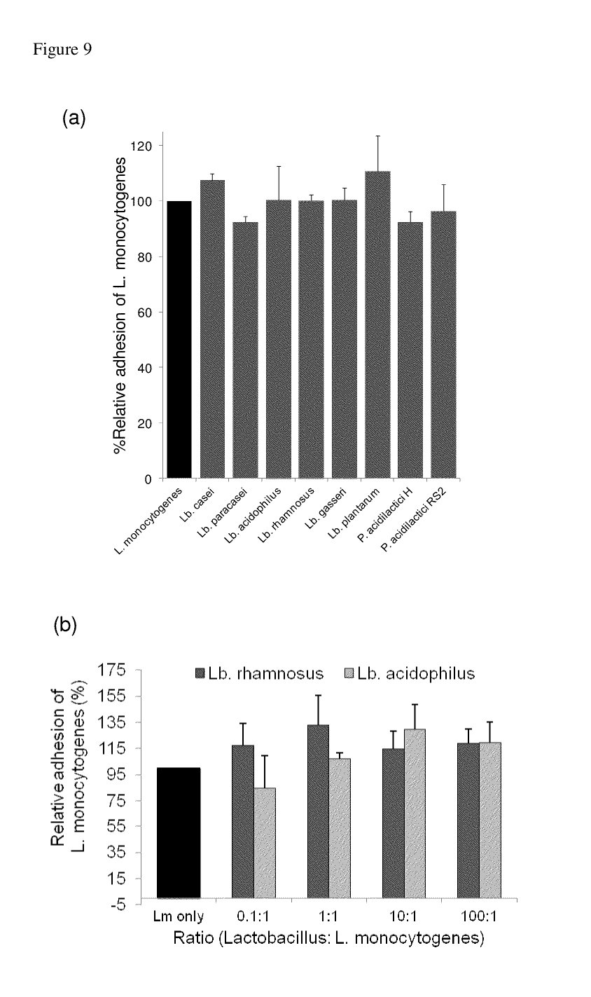

Three experimental approaches were carried out to examine whether the selected lactobacilli would reduce L. monocytogenes adhesion to Caco-2 cells: competitive exclusion, inhibition of adhesion, and displacement (FIG. 8). Surprisingly, none of the lactobacilli reduced the adhesion of L. monocytogenes at significant levels regardless of method used (FIG. 8), despite their uniform attachment to Caco-2 cells throughout the study. Five additional LAB strains also did not displace attached L. monocytogenes from Caco-2 cells (FIG. 9a). It was examined whether increased concentrations of lactobacilli could reduce L. monocytogenes adhesion. Lactobacilli added in 100-fold greater numbers also failed to displace attached L. monocytogenes (FIG. 9b). These data clearly indicated that lactobacilli and other tested LAB strains were unable to reduce or prevent L. monocytogenes adhesion or colonization on epithelial cell surfaces, even in higher numbers.

### Example 3

### LAP of L. monocytogenes Cloning and Expression in Lb. paracasei

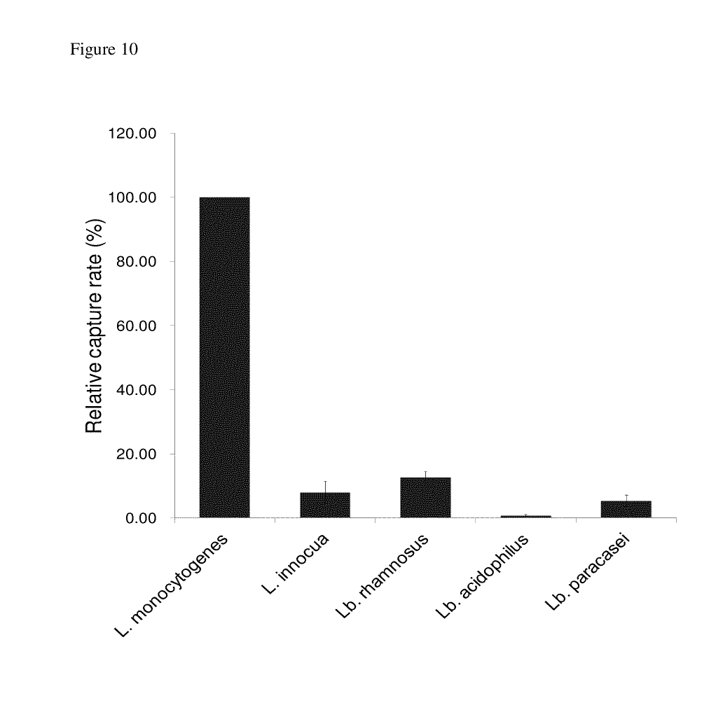

Before initiating the cloning experiment, it was verified if Lb. rhamnosus, Lb. acidophilus, and Lb. paracasei would interact with Hsp60, because they also carry a LAP homolog (Table 3); however, a magnetic bead binding experiment (FIG. 10) and a microfluidic biochip experiment revealed no apparent interaction of these lactobacilli with purified human Hsp60 protein.

Because none of the WT LAB showed any discernable inhibition of L. monocytogenes, it was sought to determine whether LAP expression in probiotic bacteria would reduce L. monocytogenes infection in the competitive exclusion experiment. First the lap gene was cloned in a Lactobacillus expression vector, pLP401-T (FIG. 2a), and transformed it into Lb. paracasei (Table 1, above), which had an intermediate level of attachment to Caco-2 cells (see FIG. 1; Note: the pLP401-T vector was originally designed for heterologous gene expression in Lb. paracasei and Lb. casei, hence Lb. paracasei was used to express L. monocytogenes LAP). Sequence similarity between LAP, an alcohol acetaldehyde dehydrogenase (Aad) from Listeria monocytogenes and Lactobacilli (Table 3).

TABLE 3
Sequence similarity between LAP, an alcohol
acetaldehyde dehydrogenase (Aad) from Listeria monocytogenes
and Lactobacilli.

| | NCBI | Identities | Positives |
|---|---|---|---|
| Bacteria | accession | (%) | (%) |
| Lactobacillus | ZP_03211500 | 60% (528/871^{a}) | 75% (659/871) |
| rhamnosus | | | |
| Lb. acidophilus | YP_193379 | 50% (443/878) | 67% (593/878) |
| Lb. paracasei | ZP_04672734 | 60% (525/872) | 75% (661/872) |

^{a}Number of identical or similar amino acids from total amino acids of Aad from L. monocytogenes

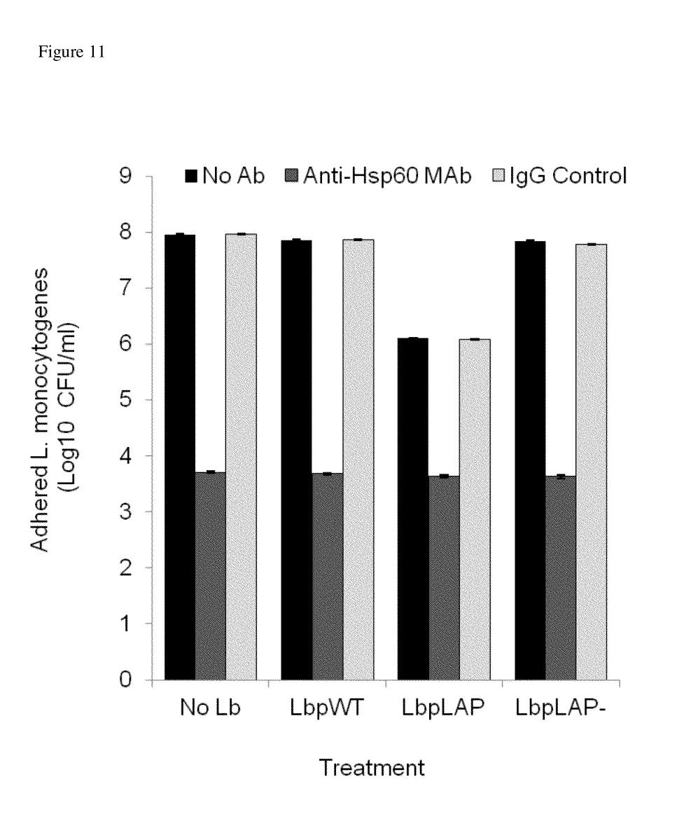

Protein expression in recombinant Lb. paracasei (Lbp^{LAP}) cell fractions was analyzed with Western blot. Data indicated that LAP was present in the supernatant (SN), cell wall (CW), and intracellular fractions (FIG. 2b). Aminopeptidase C (PepC) assay confirmed that the SN and CW fraction had no apparent contamination from intracellular proteins (data not shown). Furthermore, anti-LAP MAb EMH7 showed no reaction with protein bands from Lb. paracasei WT (Lbp^{WT}) (see FIG. 2b). These data indicated that LAP is surface associated in Lbp^{LAP} cells and would be available for interaction with mammalian cells. Additionally, immunofluorescence staining using anti-LAP MAb confirmed the surface localization (FIG. 2c). LAP interacts with mammalian protein receptor Hsp60. To verify whether surface-expressed LAP from Lbp^{LAP} would interact with Hsp60, purified mammalian Hsp60 protein was immobilized on paramagnetic beads, and the capture rate of Lbp^{LAP} cells was determined relative to L. monocytogenes capture. If the bead-based capture efficiency of L. monocytogenes WT was considered 100%, the percent relative capture for Lbp^{LAP} cells was 86.5%, which was 4.4-fold higher than that of Lbp^{WT} (19.6%; FIG. 2d). In a separate experiment, it was demonstrated that pretreatment of Caco-2 cells with anti-Hsp60 monoclonal antibody (1 μg/ml) affected Lbp^{LAP} binding and subsequently L. monocytogenes adhesion (FIG. 11). Collectively, these data confirmed that LAP of L. monocytogenes was successfully expressed in Lb. paracasei and surface-associated LAP efficiently interacted with Hsp60.

### Example 4

### LbpLAP Adherence and Translocation Through Caco-2 Cell Monolayers

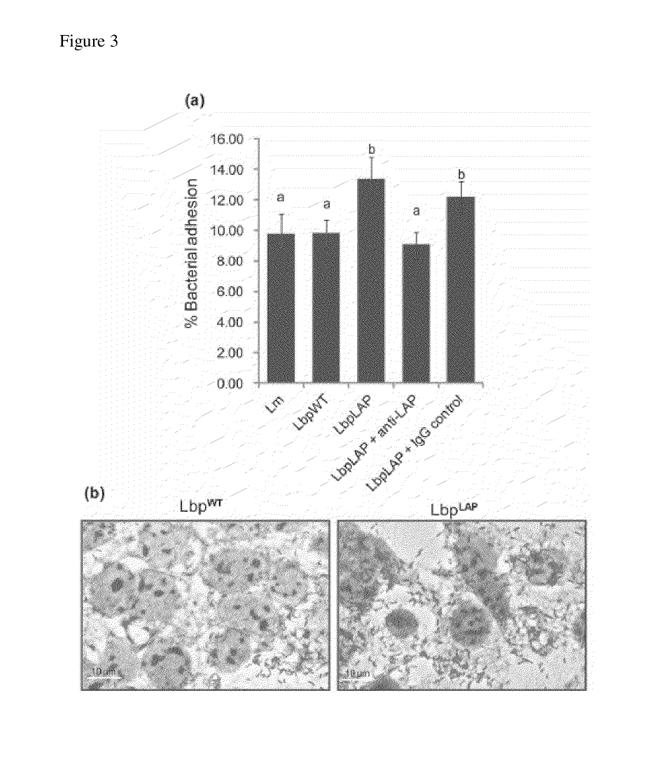

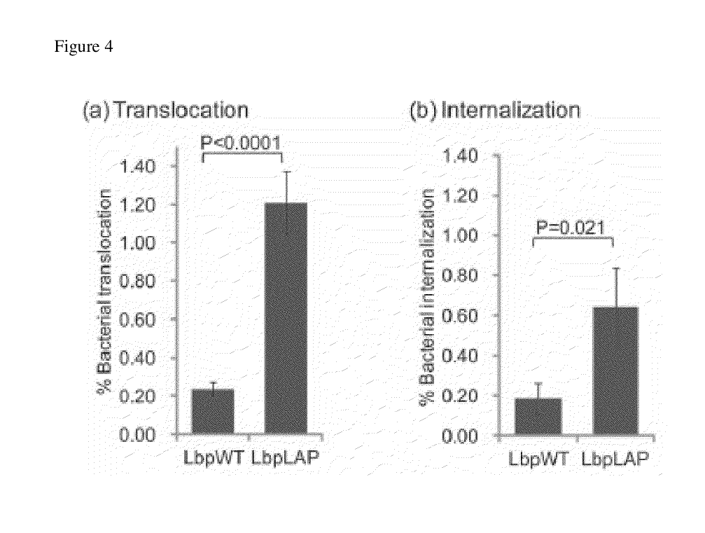

The adhesion and transepithelial translocation characteristics of recombinant Lbp^{LAP} in Caco-2 cells was also examined. The data showed a significant increase (P=0.0009) in adhesion of Lbp^{LAP} compared to Lbp^{WT} (FIG. 3a), demonstrating the involvement of LAP in adhesion. Giemsa staining of the Caco-2 cell monolayer also provided visual confirmation of qualitative increase in adhesion for Lbp^{LAP} cells (FIG. 3b). LAP involvement was further verified by pre-treatment of Lbp^{LAP} cells with anti-LAP monoclonal antibody (MAb-H7) which reduced adhesion by 4.3% compared to antibody-untreated Lbp^{LAP} cells or cells treated with isotype immunoglobulin G control antibody (FIG. 3a). Further, it was examined whether Lbp^{LAP} had translocation ability similar to that of L. monocytogenes. Using a standard transwell setup, it was demonstrated that Lbp^{LAP} cells translocated through epithelial cell monolayers with greater efficiency—i.e., 5.1-fold (P<0.0001) higher—than that of LbpWT (FIG. 4a). It was also examined the internalization of Lbp^{LAP} by Caco-2 cells. Interestingly, Lbp^{LAP} cells were internalized at about a 3.5-fold higher level than that of the Lbp^{WT} (FIG. 4b).

### Example 5

### LbpLAP Reduces L. monocytogenes Adhesion and Transepithelial Translocation Through Caco-2 Cell Monolayers

It was also investigated the ability of Lbp^{LAP} to reduce or prevent L. monocytogenes attachment to Caco-2 cells using the three competitive exclusion assays. In the competitive adhesion experiment, Caco-2 cells were exposed to Lbp^{LAP}, LbpWT, and L. monocytogenes for 1 h each before bacterial enumeration. In the competitive adhesion assay, adhesion of L. monocytogenes was reduced by 31.0% (FIG. 5a), and in the inhibition of adhesion assay, reduction was 24.6% compared to that of L. monocytogenes alone (FIG. 5b). No significant difference in displacement of L. monocytogenes occurred with Lbp^{LAP} (P=0.3147; FIG. 5c). Inhibition in adhesion of lap-deficient mutant L. monocytogenes KB208 by Lbp^{LAP} (negative control) was not observed. Overall, the recombinant strain effectively excluded L. monocytogenes when added before (inhibition of adhesion) or simultaneously (competitive adhesion) but not after L. monocytogenes has already adhered (displacement assay). The adhesion of Lbp^{LAP} cells was also monitored. These cells showed a 21.7% reduction in binding during competitive adhesion with L. monocytogenes, whereas no reduction occurred in the to displacement assay; however, Lbp^{LAP} cell adhesion was significantly reduced after the inhibition assay (44.1% reduction). Using the competitive adhesion assay, the effect of LbpLAP cell pre-exposure on Caco-2 cells was determined for 1, 4, 15, and 24 h and the reduction of L. monocytogenes infection—i.e., adhesion, invasion, and trans-epithelial translocation. The data showed that Lbp^{LAP} cells reduced L. monocytogenes adhesion by 21%, 26%, 33%, and 44%, respectively, whereas LbpWT exposure resulted in only a 3.5%-14.6% reduction during the same period (FIG. 6a). Invasion experiment showed that Lbp^{LAP} reduced L. monocytogenes invasion by 8.3%, 7.3%, 27.6%, and 44.7%, respectively (FIG. 6b). Transepithelial translocation experiments demonstrated highly significant effects against L. monocytogenes: Lbp^{LAP} reduced L. monocytogenes translocation by 15.3%, 31.8%, 36.8%, and 46.3% in 1, 4, 15, and 24 h, respectively (FIG. 6c), whereas Lbp^{WT} had no significant effect. In these experiments, a vector control, devoid of lap insert (Lbp^{LAP}-) was included to rule out the involvement of any plasmid encoded proteins (pLP401-T) that may exert protective effect against L. monocytogenes (FIG. 6). Together, these data indicate that increased preoccupation of Hsp60 on Caco-2 cells by growing Lbp^{LAP} cells overtime significantly (P<0.05) reduced L. monocytogenes adhesion, invasion, and translocation though epithelial barriers.

### Example 6

### Lbp^{LAP} Reduces L. monocytogenes-Induced Tight Junction Permeability

L. monocytogenes may alter tight junction permeability to allow for its own translocation through the epithelial barrier. Hence, Caco-2 tight junction integrity was monitored using the well established dextran fluorescein isothiocyanate (Dextran-FITC) permeability assay. After infection with L. monocytogenes for 2 h, about 2.6% of the apical Dextran^{FITC} was recovered from the basolateral chamber, indicating a compromise in tight junction integrity. In contrast, pre-exposure of Caco-2 monolayers to Lbp^{LAP} for 1-24 h before L. monocytogenes infection reduced Dextran^{FITC} recovery to 0.3% or less (Table 4), a level equivalent to that from uninfected Caco-2 cells. These data demonstrated that Lbp^{LAP} can protect Caco-2 cells from L. monocytogenes mediated cell damage and tight junction compromise. Likewise, tight junction integrity was monitored by measuring transepithelial electrical resistance (TEER; Table 5). Percent change in TEER values for Caco-2 cells pre-exposed to LbpWT followed by 2 h of treatment with L. monocytogenes varied from 8.8% to 14.5%; however, values for LbpLAP-treated cells followed by L. monocytogenes infection was only 1.4%-6.4%. These data confirm the ability of LbpLAP to prevent L. monocytogenes translocation through epithelial cell barriers, possibly by maintaining tight junction integrity (see FIG. 6).

TABLE 4
Tight junction integrity analysis with Dextran^{FITC} permeability assays.

| | % Apical Dextran^{FITC} recovered in bottom well after Caco-2 cells were | | | |
|---|---|---|---|---|
| | pretreated with Lactobacillus paracasei for variable time periods followed by | | | |
| | Listeria monocytogenes treatment for 2 h (Mean (SE))^{a} | | | |
| Treatment | 1 h | 4 h | 15 h | 24 h |
| Lb. paracasei WT (Lbp^{WT}) | 2.11 ± 0.04 | 2.28 ± 0.05 | 2.56 ± 0.07 | 2.54 ± 0.12 |
| Lb. paracasei LAP (Lbp^{LAP}) | 0.09 ± 0.01 | 0.32 ± 0.02 | 0.34 ± 0.001 | 0.34 ± 0.01 |
| Fold-change | 24.8 | 7.1 | 7.5 | 7.5 |

^{a}Caco-2 cells monolayers were grown in transwell inserts and treated with wild-type (WT) or Listeria adhesion protein (LAP)-expressing Lb. paracasei for 1, 4, 15 and 24 h, then treated with L. monocytogenes for 2 h. Tight junction integrity of Caco-2 cells was monitored with Dextran ^{FITC} translocation across the membrane. Dextran^{FITC} recovery after L. monocytogenes was 2.68 ± 0.03%. Values are averages of three experiments analyzed in triplicate and are significantly different between Lbp^{WT} and Lbp^{LAP} at all time points (P < 0.05).

[symbol]

[symbol]

indicates data missing or illegible when filed

TABLE 5
Caco-2 cell permeability analysis using transepithelial electrical resistance (TEER).

| | | | TEER (Mean Ω/cm² (SE))^{a} | | |
|---|---|---|---|---|---|
| | Exposure | | Before exposure to | After exposure to | |
| Treatment | time (h) | | Listeria monocytogenes | L. monocytogenes (2 h) | % Change |
| Lactobacillus paracasei WT (Lbp^{WT}) | 1 | h | 268.6 ± 3.9 | 244.9 ± 4.7 | 8.5 |
| | 4 | h | 269.9 ± 2.9 | 239.9 ± 2.1 | 10.8 |
| | 15 | h | 265.5 ± 3.3 | 226.9 ± 2.2 | 14.5 |
| | 24 | h | 271.4 ± 2.4 | 212.9 ± 3.1 | 14.2 |
| Lb. paracasei LAP (Lbp^{LAP}) | 1 | h | 266.5 ± 3.4 | 262.9 ± 3.1 | 1.4 |
| | 4 | h | 267.1 ± 3.5 | 261.5 ± 4.0 | 2.1 |
| | 15 | h | 263.9 ± 1.5 | 252.8 ± 0.8 | 4.3 |
| | 24 | h | 268.7 ± 4.1 | 251.5 ± 3.6 | 6.4 |

^{a}Caco-2 cells monolayers were grown in transwell inserts and treated with wild-type (WT) or Listeria adhesion protein (LAP)-expressing Lb. paracasei for 1, 4, 15 and 24 h, then treated with L. monocytogenes for 2 h. TEER measurements before and after L. monocytogenes treatment alone were 279.40 ± 1.19 and 243.87 ± 1.20, respectively. Values are averages of two experiments analyzed in triplicate and are significantly different between Lbp^{WT} and Lbp^{LAP} at all time points (P < 0.05). % Change was calculated as 1 − TEER_{after} + TEER_{before} × 100.

[symbol]

[symbol]

indicates data missing or illegible when filed

### Example 7

### Lbp^{LAP} Reduces L. monocytogenes-Induced Cell Cytotoxicity

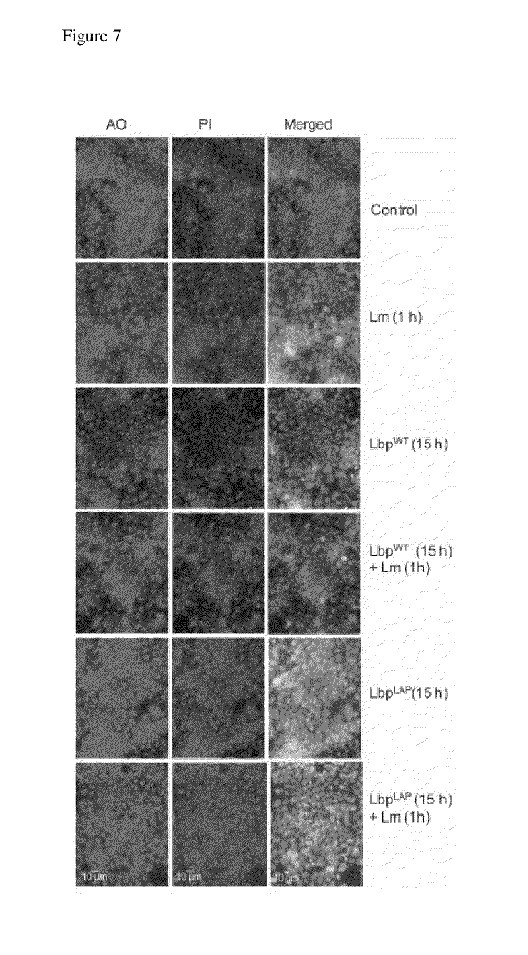

L. monocytogenes induces severe cell cytotoxicity in mammalian Cells. It was examined whether Lbp^{LAP} could protect Caco-2 cells from this cytotoxicity. Lactate dehydrogenase assay indicated that LbpLAP reduced L. monocytogenes-mediated cytotoxicity by 99.8% after 1 h of pre-exposure, 88.8% after 4 h, 80% after 15 h, and 79% after 24 h, whereas Lbp^{WT} demonstrated no discernable protective effects (Table 6). Reduced Lbp^{LAP}-mediated protection after 15 and 24 h of pre-exposure may be due to the overgrowth of Lbp^{LAP} and consequent production of metabolic by-products with adverse effects on Caco-2 cells, which make them more vulnerable to L. monocytogenes-mediated cell damage. Under in vivo to conditions, these by-products would be processed by luminal cells or natural microflora. Reduced cytotoxicity was also verified with live and dead staining of Caco-2 cells using acridine orange (AO) and propidium iodide (PI). L. monocytogenes-infected aco-2 cells pretreated with and without Lbp^{WT} for 15 h appeared orange-red, indicating that the majority of cells were either dead or their cell membranes were severely compromised. When the Caco-2 cells were pre-exposed to Lbp^{LAP} before L. monocytogenes infection, however, they appeared bright green, indicating that they were similar to uninfected controls (FIG. 7).

TABLE 6
Cytotoxicity of Listeria monocytogenes on Caco-2 cells pretreated with Lactobacillus paracasei.

| | % Cytotoxicity induced by L. monocytogenes to Caco-2 cells pretreated with | | | |
|---|---|---|---|---|
| | Lb. paracasei for variable time periods (Mean (SE))^{a} | | | |
| Treatment | 1 h | 4 h | 15 h | 24 h |
| Lb. paracasei WT (Lbp^{WT}) | 56.9 ± 0.14 | 59.0 ± 0.7 | 61.6 ± 0.8 | 65.3 ± 0.9 |
| Lb. paracasei LAP (Lbp^{LAP}) | 0.09 ± 0.02 | 7.4 ± 1.5 | 12.7 ± 0.3 | 13.7 ± 0.6 |
| % Protection | 99.8 | 88.8 | 80 | 79 |

^{a}Lb. paracasei cultures were added to Caco-2 cells at a multiplicity of exposure (MOE) of 10:1 for 1, 4, 15, and 24 h before infection with L. monocytogenes (MOI of 10:1) for 1 h. Cytotoxicity value for L. monocytogenes alone was 66.21 ± 3.1. Values are averages of three experiments analyzed in triplicate and are significantly different between Lbp^{WT} and Lbp^{LAP} at all time points (P < 0.05).

[symbol]

[symbol]

indicates data missing or illegible when filed

### Example 8

### Listeria Adhesion Protein from Non-Pathogenic L. innocua Restored Adhesive Property of LAP-Deficient L. Monocytogenes to Enterocytes

LAP from pathogenic Listeria is structurally similar to that from nonpathogenic Listeria. However, LAP-mediated adhesion does not occur in non-pathogen (i.e., L. innocua) due to a defect in surface re-association mechanism. Thus LAP from L. innocua was cloned and expressed in a lap-mutant of L. monocytogenes KB208. Adhesion analysis revealed that KB208 expressing LAP of L. innocua F4248 (AKB308) adhered to Caco-2 cells, at similar levels as the WT L. monocytogenes. Hence, expression of LAP from a non-pathogen in probiotics is a novel approach to avoid any potential concern of using a gene from a pathogenic strain for human use.

### Example 9

### LAP of Listeria innocua was Successfully Expressed in Recombinant Lactobacillus casei

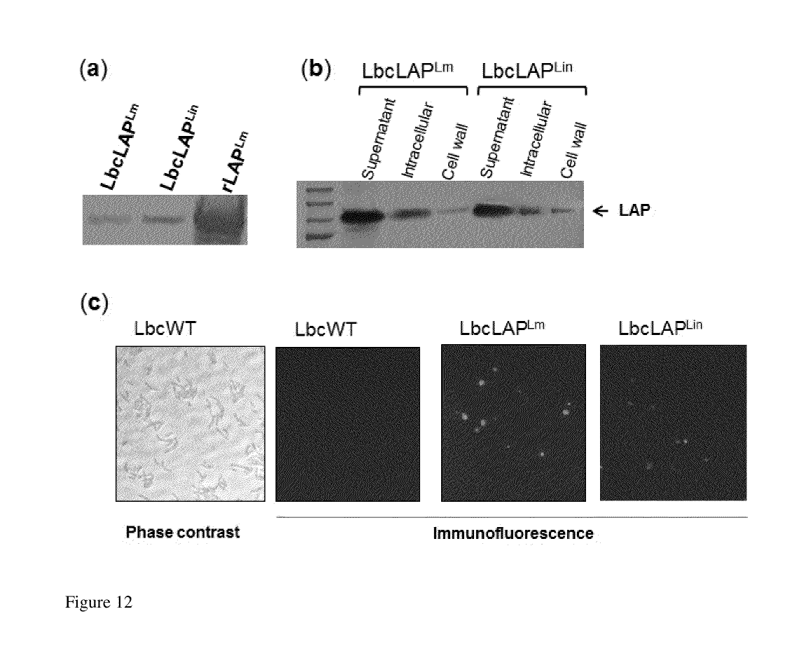

The lap gene from L. innocua and L. monocytogenes was first cloned into a Lactobacillus expression vector, pLP401T, and transformed it into Lactobacillus casei (Table 2). Protein expression in recombinant Lb. casei expressing LAP of L. innocua (LbcLAP^{Lin}) or L. monocytogenes (LbcLAP^{Lm}) cell fractions was analyzed by Western blotting. Data indicated that to LAP was present in the supernatant (SN), cell wall (CW), and intracellular fractions (FIG. 12a-b). Phosphoenolpyruvate carboxylase (PepC) assay confirmed that the SN and CW fraction had no apparent contamination from intracellular proteins (data not shown). Furthermore, anti-LAP MAb-H7 showed no reaction with protein bands from Lb. casei WT (Lbc^{WT}) (data not shown). This study indicated that LAP is surface associated in recombinant LbcLAP^{Lin} and LbcLAP^{Lmn} strains and would be available for interaction with mammalian cells. Additionally, immunofluorescence staining using anti-LAP MAb confirmed LAP surface localization on bioengineered probiotics (FIG. 12c).

### Example 10

### Recombinant Bioengineered Probiotic Lb. casei Expressing LAP of L. innocua (LbcLAP^{Lin}) Reduced Adhesion and Translocation of L. monocytogenes Through Epithelial Barrier

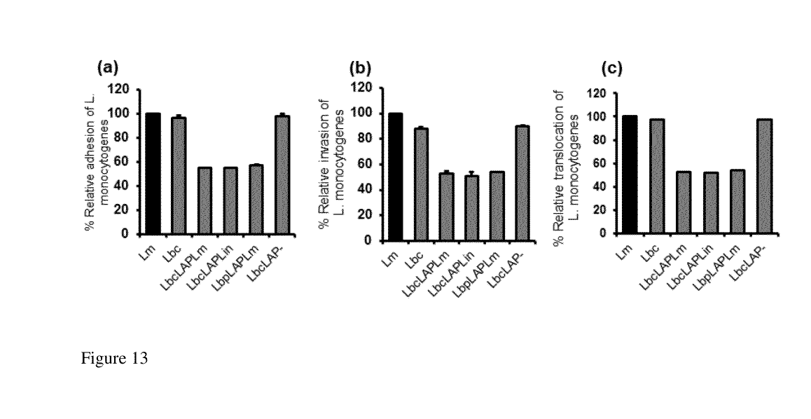

Using the competitive exclusion assay, we determined the effect of pre-exposure of LbcLAP^{Lin} and LbcLAP^{Lm} to Caco-2 cells for 24 h and the reduction of L. monocytogenes infection—i.e., adhesion, invasion, and transepithelial translocation. The data showed that LbcLAP^{Lin} cells reduced L. monocytogenes adhesion, invasion and translocation counts by 99%, whereas Lbp^{WT} exposure resulted in only a 36.9, 73.3 and 30.8% reduction, respectively, during the same period (FIG. 13, Table 7). Pre-exposure to LbcLAP^{Lm} also resulted in a reduction in L. monocytogenes populations similar to LbcLAP^{Lin}. In all these experiments, a plasmid control (LbcLAP⁻) was included to exclude any effects that could be attributed to any other component associated with the plasmid. The recombinant LbcLAP⁻ strain reduced L. monocytogenes populations similar to LbcWT thus demonstrating that the protective effect observed with LbcLAP^{Lin} are specifically due to LAP insertion. Together, these data indicate that occupation of receptor site (Hsp60) on Caco-2 cells by LbcLAP^{Lin} and LbcLAP^{Lm} cells significantly reduced L. monocytogenes adhesion, invasion, and transepithelial translocation.

TABLE 7

| | L. monocytogenes counts (log10 CFU/ml) ± SEM^{a} | | |
|---|---|---|---|
| Treatments | Adhesion | Invasion | Translocation |
| L. monocytogenes F4244 (WT) | 5.77 ± 0.07 | 5.39 ± 0.13 | 5.54 ± 0.17 |
| Lb. casei WT (LbcWT) | 5.57 ± 0.13 | 4.80 ± 0.05 | 5.38 ± 0.01 |
| | (36.9) | (74.30) | (30.82) |
| Lb. caseiLAP^{Lm} (LbcLAP^{Lm}) | 3.17 ± 0.01 | 2.85 ± 0.09 | 2.91 ± 0.01 |
| | (99.75) | (99.71) | (99.77) |
| Lb. caseiLAP^{Lin} (LbcLAP^{Lin}) | 3.16 ± 0.01 | 2.75 ± 0.16 | 2.87 ± 0.01 |
| | (99.75) | (99.77) | (99.78) |
| Lb. paracaseiLAP^{Lm} (LbpLAP^{Lm})^{b} | 3.29 ± 0.05 | 2.93 ± 0.01 | 2.99 ± 0.01 |
| | (99.67) | (99.65) | (99.72) |
| Lb. casei LAP- (LbcLAP⁻)^{c} | 5.66 ± 0.1 | 4.86 ± 0.04 | 5.38 ± 0.01 |
| | (22.38) | (70.49) | (30.82) |

^{a}Caco-2 cells were pre-exposed to probiotics for 24 h before challenged with L. monocytogenes (MOI: 10:1) for 1-2 h and assayed for L. monocytogenes counts. Values in parenthesis indicate percent reduction in L. monocytogenes counts after treatment with probiotics compared to the positive control, L. monocytogenes WT treated Caco-2 cells.
^{b}This recombinant strain carrying LAP was created previously in our lab and was used as a control (Koo et al. 2012).
^{c}This strain contained the empty vector, pLP401T without any insert

### Example 11

### Recombinant Probiotic Survival was Unaffected by Simulated Gastric and Intestinal Fluid

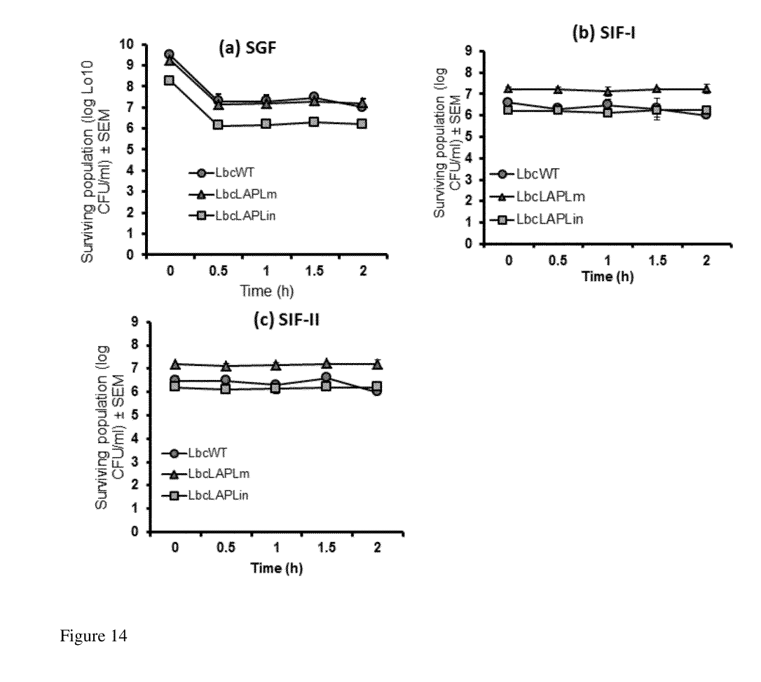

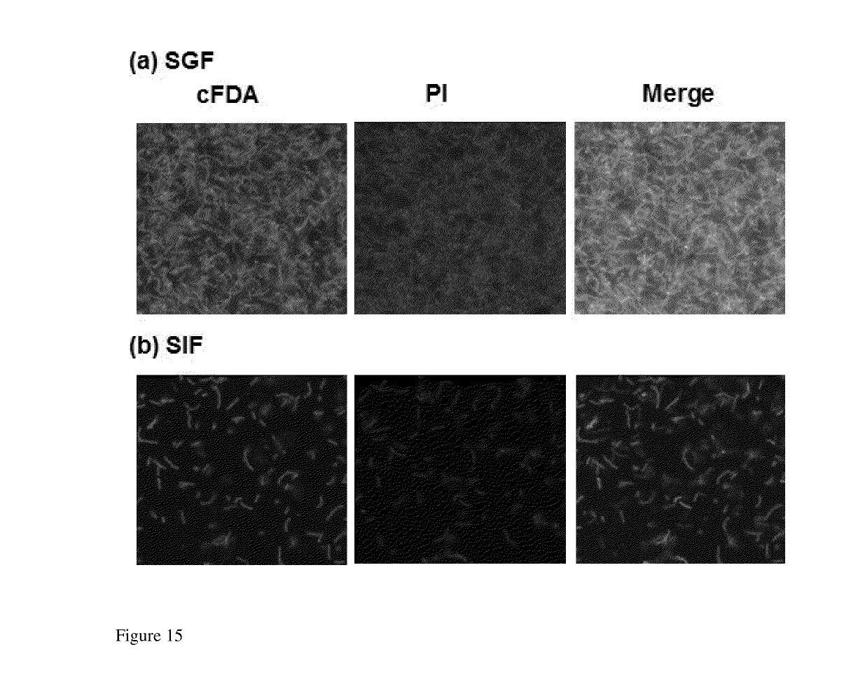

It is essential to determine survival of wild-type and recombinant Lb. casei in simulated gastric and enteric conditions during transit through GI tract. The probiotic cultures were initially exposed to simulated gastric fluid (SGF) at pH of 1.2-1.5 for a period of 2 h. Exposure to low pH resulted in an initial reduction in probiotic population by 2.5 log after 30 min following which the bacterial population remained stable with a final count of 6.5-7.5 log by the end of 2 h of incubation (FIG. 14). Following the gastric phase, the probiotics were exposed to simulated intestinal fluids (SIF-I: pH 4.3-5.2) and SIF-II: pH 6.7-7.5) mimicking the small and large intestinal fluids, respectively, and these did not significantly (P>0.05) affect probiotic survival. Live and dead staining of probiotics exposed to SGF and SFI also confirmed probiotic survival (FIG. 15). These data ensured survival of the wild type or bioengineered probiotics during transit through and residence in the gastrointestinal environment of the host.

Survival of probiotics in deionized water was also monitored for 24 h at room temperature since probiotics suspended in water were supplied to mice in bottle every day. Overall probiotics counts at 0 h varied from 5.47 to 6.74 log CFU/ml and at 24 h the counts were 6.1 to 6.38 log CFU/ml indicating probiotics maintained same counts in water for 24 h.

### Example 12

### Recombinant Lb. casei Prevented or Reduced Extra-Intestinal Dissemination of L. monocytogenes in a Mouse Model

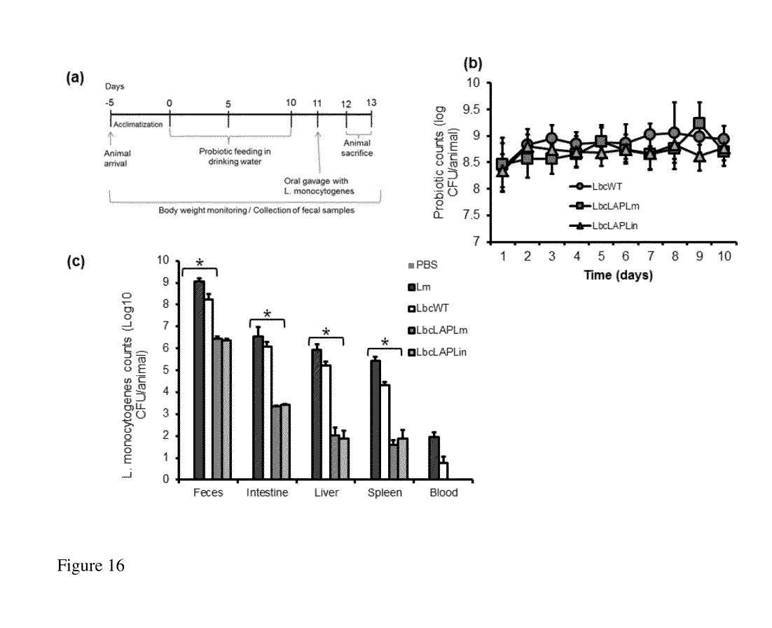

To examine the efficacy of recombinant probiotic in protecting mice from L. monocytogenes infection, extra-intestinal dissemination of bacteria to blood, liver and spleen were monitored. Female A/J mice acclimatized for five days were fed with LbcWT, LbcLAP^{Lm} and LbcLAP^{Lin} in drinking water for ten days prior to oral gavage with L. monocytogenes and sacrificed after 24 and 48 h (FIG. 16a).

First, probiotic colonization in the mice for 10-days of feeding was monitored by enumerating fecal shedding of probiotics daily on MRS agar plates containing antibiotics. Data to show both wild type and recombinant probiotic counts were in the range of 8.4-8.7 log CFU/mouse and provided indirect evidence for probiotic colonization in the intestine over a 10 day period (FIG. 16b). As expected, control groups did not give any recombinant probiotic counts.

Protective effects of probiotics against L. monocytogenes infection was monitored by analyzing the pathogen translocation from intestine to extra-intestinal tissues (liver, spleen, blood) of mice (Corr et al. 2007; Lecuit et al. 2001). L. monocytogenes counts in extra-intestinal tissues in mice fed with recombinant probiotic for 10 days were significantly (P<0.05) lower than the mice fed with wild type probiotic or no probiotics at all (Table 8, FIG. 16c). Recombinant probiotic (LbcLAP^{Lin}) reduced L. monocytogenes counts in liver by 4.05 log (99.99%), spleen by 3.55 log(99.97%) and 100% in blood (undetectable) compared to animals that did not receive any probiotics. Similar extraordinary protection was also noticed when mice were fed with recombinant probiotic carrying LAP of L. monocytogenes (LbcLAP^{Lm}). Interestingly, reduction in L. monocytogenes counts by wild type probiotic (LbcWT) was negligible showing only 0.76 log(82%) reduction in blood, 0.72 log(81%) in liver and 1.12 log (92.4) in spleen (Table 8, FIG. 16c). Furthermore, the recombinant probiotics also significantly lowered L. monocytogenes counts in intestines and feces. These results are in agreement with our in vitro cell culture experiment (FIG. 13). Collectively, these data provide strong evidence that LAP from a nonpathogenic L. innocua strain expressed in recombinant probiotic is able to protect animals against L. monocytogenes infection similar to recombinant probiotic expressing LAP of L. monocytogenes strain.

Further analysis revealed that among the wild type probiotic fed-animals, L. monocytogenes was recovered from liver and spleen from all animals (10/10) at concentrations ranging from 4.31-5.2 log CFU/mouse (Table 8). Clinically, these animals appeared sick exhibiting ruffled hair, recumbence, reduced responsiveness and movement, and refrained from feed and drink. While L. monocytogenes was recovered only from 50% (10/20) of the recombinant probiotics (LbcLAP^{Lin} and LbcLAP^{Lm})— fed mice, and bacterial loads were only at 1.6-2 log CFU/mouse. A majority (99%) of these animals appeared healthy and continued to feed and drink (Table 8).

TABLE 8

| | | | | | | | Mice | |
|---|---|---|---|---|---|---|---|---|
| | | | | | | | positive/total | |
| | | | | | | | for | |
| | | L. monocytogenes counts | | | | | Lm in | Clinical |
| | | (log10 CFU/mouse) ± SEM^{a} | | | | | liver and | signs |
| Treatment | Mice | Blood | Liver | Spleen | Intestine | Feces | spleen^{c} | (sick/total)^{d} |
| Control^{b} | 18 | ND | ND | ND | ND | ND | 0/18 | 0/18 |
| L. mono | 10 | 1.542 ± 0.21 | 5.92 ± 0.25 | 5.43 ± 0.19 | 6.54 ± 0.44 | 9.06 ± 0.16 | 10/10 | 10/10 |
| F4244 (WT) | | | | | | | | |
| Lb. | 10 | 0.78 ± 0.29 | 5.20 ± 0.20 | 4.31 ± 0.16 | 6.1 ± 0.23 | 8.22 ± 0.26 | 10/10 | 10/10 |
| casei ATCC344 | | (81.79) | (80.95) | (92.41) | (63.70) | (85.55) | | |
| (WT) | | | | | | | | |
| Lb. | 10 | ND | 2.0 ± 0.36 | 1.58 ± 0.22 | 3.34 ± 0.04 | 6.42 ± 0.11 | 5/10 | 0/10 |
| casei PL LAP^{Lm} | | (100) | (99.99) | (99.99) | (99.94) | (99.77) | | |
| Lb. | 10 | ND | 1.87 ± 0.35 | 1.88 ± 0.39 | 3.43 ± 0.03 | 6.36 ± 0.08 | 5/10 | 1/10^{e} |
| casei LAP^{Lin} | | (100) | (99.99) | (99.97) | (99.92) | (99.80) | | |

^{a}Values in parenthesis indicate percent reduction in L. monocytogenes counts in organs and tissues compared to the positive control (i.e., mice received L. monocytogenes WT only).
^{b}Control group includes animals that were fed only PBS/LbcWT/LbcLAP^{Lm}/LbcLAP^{Lin} and no L. monocytogenes challenge.
^{c}L. monocytogenes(Lm) negative tissues were also negative even after enrichment in Listeria-selective enrichment broth, Fraser Broth (FB) for 24 h.
^{d}Animals were less responsive to external stimuli, no voluntary movement, and they appeared hunched with ruffled hair. Sick animals had also very low fecal outputs and refrained from feed and water.
^{e}The sick animal in this group had L. monocytogenes counts in liver and spleen 3.72 log CFU/mouse and 4.63 log CFU/mouse, respectively and it was thought to be infected with a pathogen other than L. monocytogenes.

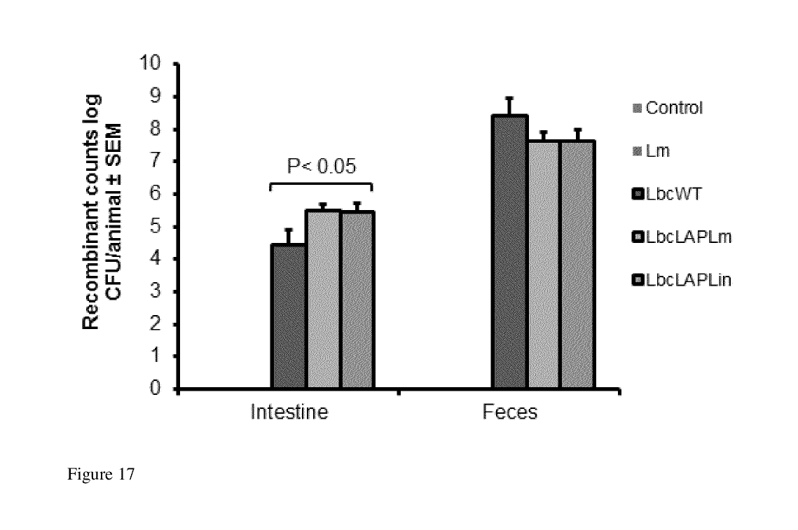

Both wild type and recombinant probiotics counts were also monitored in the extra-intestinal tissues, intestine and feces on day 12. Probiotics were not isolated from any extra-intestinal sites (liver, spleen and blood) thus indicating that the recombinant probiotics were either unable to cross epithelial barrier or the translocated bacteria were quickly cleared up by the immune system. This certainly rejects the concerns of unintended systemic spread of the recombinant probiotics. As expected, in the intestine, recombinant probiotics (LbcLAP^{Lin} and LbcLAP^{Lm}) counts (5.46-5.5 log CFU/mouse) were significantly higher than the LbcWT (4.42 log CFU/mouse), indicating possible LAP-mediated enhanced colonization by recombinant probiotics (FIG. 17). Corresponding fecal counts were complimentary to the intestinal counts showing numerically higher LbcWT counts (8.4 log CFU/mouse) than the recombinant counts (7.62 log CFU/mouse). These data clearly show that the recombinant strains were able to colonize the intestine at a level that was successful in preventing L. monocytogenes translocation at significant numbers through epithelial barrier and the subsequent systemic spread.

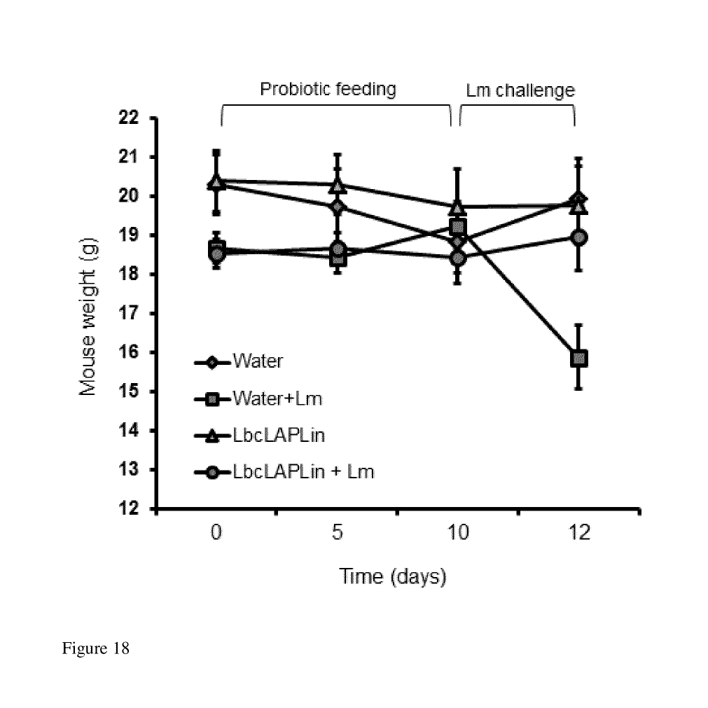

Body weight of animals was also monitored throughout the study (FIG. 18). Overall all (probiotic or non-probiotic fed) animals maintained similar body weight during the 10 days of feeding study without any significant gain in bodyweight. However, after L. monocytogenes challenge on day 10, the body weight of animals that did not receive probiotics were significantly lower (P<0.05) than the animals that received either wild type or the recombinant probiotics (FIG. 18). It was expected since recombinant probiotic fed animals prevented L. monocytogenes infection. Moreover, probiotic in general is known to promote health, thus even in wild type probiotic fed mice, L. monocytogenes did not affect the body weight.

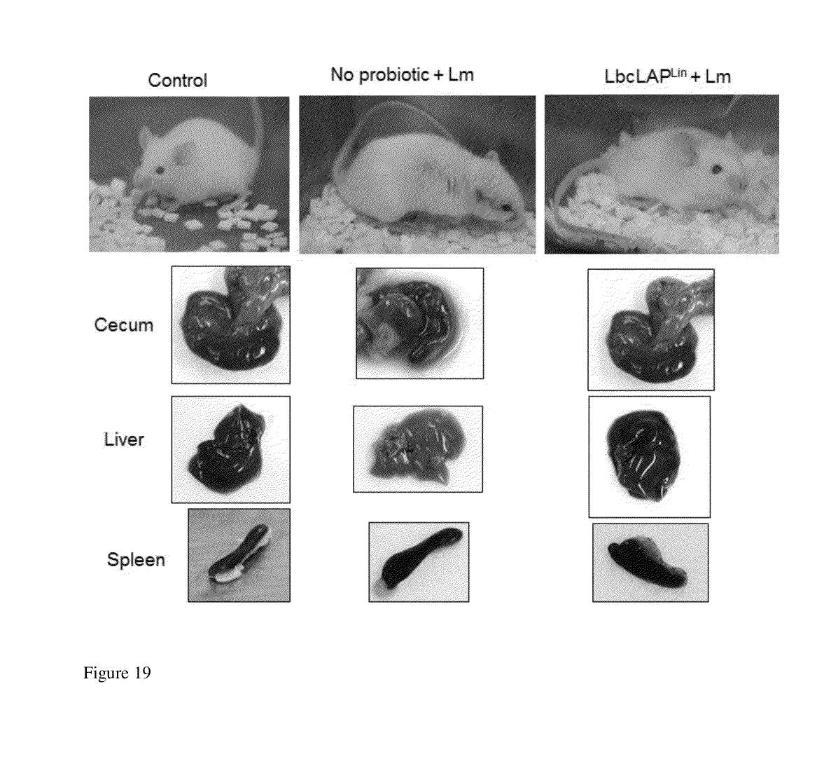

In the mouse feeding experiment, LbcLAP^{Lin} either completely prevented extra-intestinal dissemination of L. monocytogenes to liver or spleen in 50% of the mice or reduced bacterial numbers by 1,000-100,000 folds (99.9%) and the animals appeared healthy. Recombinant LbcLAP^{Lm} also exhibited similar protection against L. monocytogenes. The to appearance of liver and spleen of recombinant probiotic fed animals were similar to control uninfected group while the L. monocytogenes infected animals showed pale liver and enlarged spleen (FIG. 19). Histopathological staining of ileal tissues also did not show any signs of inflammation or tissue damage (data not shown). The recombinant probiotic mediated protection against L. monocytogenes observed in this study is extraordinary, causing a reduction of 3.5-4 log CFU/organs, especially for the recombinant strain expressing LAP that originates from a non-pathogen.

Hence, recombinant probiotics were not detected in organs and tissues implying that they are safe even though these strains are expressing listerial adhesion/translocation factor. These strains were either unable to cross the intestinal epithelial barrier or were destroyed quickly by the immune system upon translocation Animals fed with the probiotic maintained a constant probiotics count in the intestine and recombinant probiotics had significantly higher colonization than the wild type probiotics due the presence of LAP. Probiotics also did not affect body weights. These findings provide strong evidence for potential application of recombinant bioengineered probiotics for targeted prevention of listeriosis.

### Example 13

### Organs and Tissues of Recombinant Probiotic Treated Mice Did not Exhibit any Discernible Gross Changes after L. Monocytogenes Challenge

Organs and tissues harvested after animal sacrifice were also examined for gross pathological changes (FIG. 19). Pale liver and enlarged spleen were noticed in mice that were challenged with L. monocytogenes without prior exposure to probiotic compared to the control animals that did not receive either probiotics or L. monocytogenes. Interestingly, the animals that received recombinant probiotic LbcLAP^{Lin} or LbcLAP^{Lm}, the cecum, liver and spleen appeared healthy and normal compared to the control group indicating anti-infective effects of recombinant probiotic during L. monocytogenes infection.

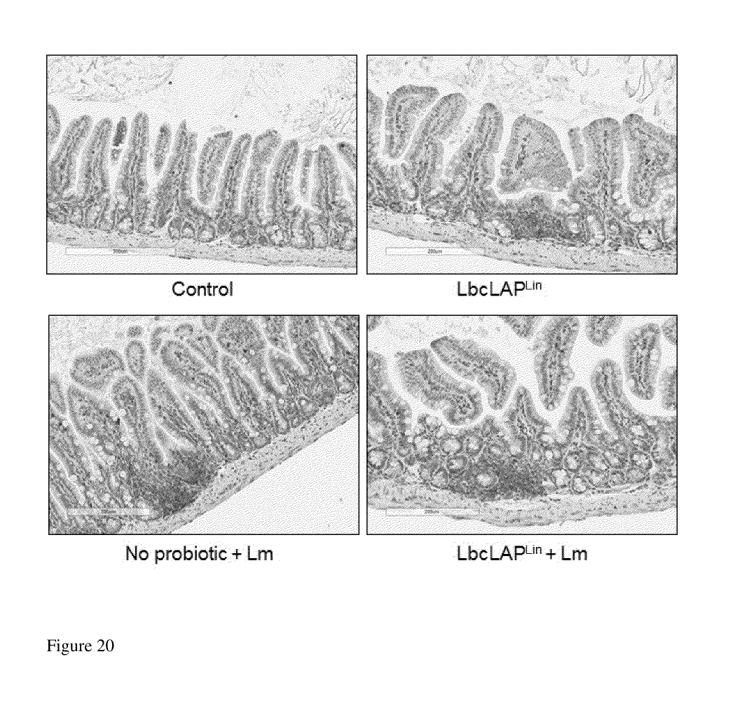

Furthermore, immunohistopathology studies did not show any significant changes in intestinal tissues (FIG. 20).

### Example 14

### Secretory IgA Level Increased after Probiotic Feeding

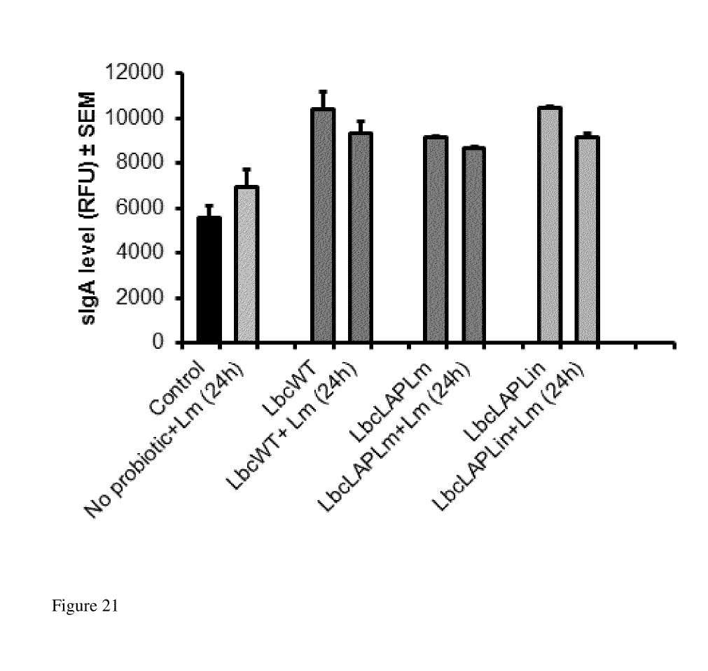

Secretory IgA levels in mice were also examined to determine if probiotics induced antibody were responsible for protecting mice against L. monocytogenes challenge. Data show that both wild type probiotic (LbcWT) and recombinant probiotics (LbcLAP^{Lm} and LbcLAP^{Lin}) induced sIgA secretion in mucus and there were no significant difference between the two treatments (FIG. 21) indicating that the role of sIgA may be minimal in protecting mice against L. monocytogenes. However, sIgA levels for probiotic treated animals were significantly higher than the probiotic untreated animals.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A recombinant probiotic bacteria strain expressing Listeria adhesion protein (LAP).

2. The probiotic bacteria of claim 1, wherein said probiotic bacteria is a LAP expressing Lactobacillus casei (Lbc-LAP) or a LAP expressing Lactobacillus paracasei (Lp-LAP).

3. The probiotic bacteria of claim 2, wherein said LAP is normally expressed from a non-pathogenic bacterial strain.

4. The probiotic bacteria of claim 3, wherein said bacterial strain is a non-pathogenic Listeria innocua strain.

5. The recombinant probiotic bacteria of claim 1, wherein said LAP is expressed on the surface of said probiotic bacteria.

6. The recombinant probiotic bacteria of claim 1, wherein said LAP binds to or interacts with mammalian protein receptor heat shock protein 60 (Hsp60).

7. The recombinant probiotic bacteria of claim 1, wherein said bacteria adhere to or colonize epithelial cells in said subject.

8. The recombinant probiotic bacteria of claim 7, wherein said epithelial cells are intestinal epithelial cells.

9. The recombinant probiotic bacteria of claim 8, wherein said epithelial cells are human intestinal epithelial cells.

10. The recombinant probiotic bacteria of claim 7, wherein said bacteria prevents adhesion, transepithelial translocation or cell cytotoxicity of LAP-expressing pathogenic bacteria into said epithelial cells.

11. The recombinant probiotic bacteria of claim 10, wherein said pathogenic bacteria is a Listeria monocytogenes strain.

12. A method of preventing a LAP-expressing pathogenic bacterial infection in a subject, the method comprising the step of administering to the subject a prophylactically-effective dose of an LAP-expressing probiotic bacterial strain, thereby preventing a LAP-expressing pathogenic bacterial infection in the subject.

13. The method of claim 12, wherein said pathogenic bacteria is Listeria monocytogenes.

14. The method of claim 12, wherein said probiotic bacteria strain is a LAP expressing Lactobacillus casei (Lbc-LAP) or a LAP expressing Lactobacillus paracasei (Lp-LAP).

15. The method of claim 12, wherein said LAP is expressed on the surface of said probiotic bacteria.

16. The method of claim 12, wherein said LAP binds to or interacts with mammalian protein receptor heat shock protein 60 (Hsp60).

17. The probiotic bacteria of claim 12, wherein said LAP is normally expressed from a non-pathogenic bacterial strain.

18. The probiotic bacteria of claim 17, wherein said non-pathogenic bacterial strain is a Listeria innocua strain.

19. The method of claim 12, wherein said subject is an immunocompromised human, a pregnant woman, a child, a human undergoing chemotherapy, or an elderly human.

20. The method of claim 12, wherein said administering is carried out via the oral route.

21. The method of claim 12, wherein said method prevents adhesion and invasion of LAP-expressing pathogenic bacteria into a cell in a subject

22. The method of claim 21, wherein said probiotic bacteria further prevents translocation of said pathogenic bacteria to said cell.

23. The method of claim 22, wherein said cell is an epithelial cell.

24. The method of claim 23, wherein said epithelial cell is an intestinal epithelial cell.

25. The method of claim 22, wherein said translocation is transepithelial translocation or paracellular translocation.

26. The method of claim 12, wherein said method prevents LAP-expressing pathogenic bacteria-induced tight junction permeability of a host cell in a subject.

27. The method of claim 26, wherein said probiotic bacteria further prevent translocation of or cell cytotoxicity of said pathogenic bacteria to said cell.

28. The method of claim 27, wherein said cell is an epithelial cell.

29. The method of claim 28, wherein said epithelial cell is an intestinal epithelial cell.

30. The method of claim 27, wherein said translocation is transepithelial translocation or paracellular translocation.

31. The method of claim 12, where said method prevents LAP-expressing pathogenic bacteria-induced cell cytotoxicity.

32. A method of delivering a foreign protein into a host cell in a subject, the method comprising administering to the subject a recombinant LAP-expressing probiotic strain comprising or expressing said foreign protein, thereby delivering a foreign protein into a host cell in the subject.

33. The method of claim 32, wherein said probiotic bacteria strain is a LAP expressing Lactobacillus casei (Lbc-LAP) or a LAP expressing Lactobacillus paracasei (Lp-LAP).

34. The method of claim 32, wherein said LAP is expressed on the surface of said probiotic bacteria.

35. The method of claim 32, wherein said LAP binds to or interacts with mammalian protein receptor heat shock protein 60 (Hsp60).

36. The probiotic bacteria of claim 32, wherein said LAP is normally expressed from a non-pathogenic bacterial strain.

37. The probiotic bacteria of claim 36, wherein said non-pathogenic bacterial strain is a Listeria innocua strain.

38. The method of claim 32, wherein said host cell is an epithelial cell.

39. The method of claim 38, wherein said epithelial cell is an intestinal epithelial cell.

40. The method of claim 32, wherein said subject is an immunocompromised human, a pregnant woman, a child, a human undergoing chemotherapy, or an elderly human.

41. The method of claim 32, wherein said administering is carried out via the oral route.
